(19) United States
(12) Patent Application Publication
(10) Pub. No.: US 2019/0037019 A1
(43) Pub. Date: Jan. 31, 2019

(54) AGENT FOR HEALTHCARE DATA APPLICATION DELIVERY

(71) Applicant: UNIVERSITY OF UTAH RESEARCH FOUNDATION, Salt Lake City, UT (US)

(72) Inventors: Darren L. Wesemann, North Salt Lake, UT (US); Michael R. Donnelly, Salt Lake City, UT (US); Orland Kip Williams, Sandy, UT (US); Cary J. Martin, Kaysville, UT (US); Ming-Chieh Tu, Salt Lake City, UT (US); Charlton Park, Holladay, UT (US); Jeffrey Todd Young, West Valley City, UT (US); Andrew Mark Hansen, Taylorsville, UT (US); Jonathan Paul Godfrey, Salt Lake City, UT (US); Nic Holbrook, Brigham City, UT (US); Timothy John Morris, Bountiful, UT (US); Tony Drake, West Jordan, UT (US); Michael Houston, Kaysville, UT (US)

(21) Appl. No.: 16/152,082

(22) Filed: Oct. 4, 2018

Related U.S. Application Data
(63) Continuation of application No. 14/723,364, filed on May 27, 2015, now abandoned.

(51) Int. Cl. H04L 29/08; G06F 19/00; G16H 10/60; G16H 15/00; H04W 4/02

(52) U.S. Cl. CPC H04L 67/1095; G06F 19/00; H04W 4/02; G16H 15/00; G16H 10/60

(57) ABSTRACT

In an example implementation, the method includes automatically determining whether a computer system is located on-premises of a health service provider or on a multi-tenant cloud. The method includes communicatively coupling with health service provider data sources via a local network and extracting health service provider data from them. The health service provider data may include protected health information (PHI) and non-PHI data. The method includes storing the PHI data and the non-PHI data in an on-premises operational data store that is located on-premises of the health service provider. The method includes obtaining data analytics based on the PHI data and the non-PHI data stored in the on-premises operational data store. The method also includes communicatively coupling with a multi-tenant cloud via a global network and synchronizing the non-PHI data in the on-premises operational data store with the multi-tenant cloud via the global network.

### CROSS-REFERENCE TO RELATED APPLICATIONS

This application is a continuation of U.S. patent application Ser. No. 14/723,364 filed on May 27, 2015, entitled “AGENT FOR HEALTHCARE DATA APPLICATION DELIVERY,” which is incorporated herein by reference in its entirety.

### FIELD

The embodiments described in this disclosure relate to data analytics for healthcare.

### BACKGROUND

Improving healthcare value is a challenge for healthcare systems of the United States and countries abroad. One measure of evaluating healthcare value is health outcomes achieved per dollar spent. Some research suggests the United States spends approximately $9000 per capita on health care annually, accounting for approximately 18% of the gross domestic product. Despite these expenditures, health outcomes are relatively poor. For example, a study reports that an estimated 440,000 Americans die prematurely each year due to preventable medical harm. The lack of correlation between spending and outcomes is fueling a national focus on value. Increasingly, healthcare payors are adopting payment models that provide strong financial incentives for the delivery of high-value care.

Payment models may offer a fixed fee for managing a population or episode of care rather than a variable fee that increases as more services are provided. Employers are also driving change. Large corporations have begun to steer high-cost, high-margin care such as cardiac and spine surgery to a small number of hospitals with demonstrated high value. Consequently, healthcare systems are faced with financial and existential imperatives to understand and improve care value.

The claimed subject matter is not limited to embodiments that solve any disadvantages or that operate only in environments such as those described above. This background is only provided to illustrate examples of where the present disclosure may be utilized.

### BRIEF SUMMARY

The present disclosure generally relates to systems for value driven outcomes to understand and improve healthcare value. The systems may be rapidly implemented and iteratively enhanced to assist healthcare organizations in evaluating costs relative to outcomes to support value improvement. The systems may facilitate healthcare organizations to maintain Health Insurance Portability and Accountability Act (“HIPAA”) compliance and/or take additional measures to secure protected health information (“PHI”) data.

In an example implementation, a method includes automatically determining whether a computer system is located on-premises of a health service provider or on a multi-tenant cloud. The method includes communicatively coupling with one or more health service provider data sources via a local network. The method includes extracting health service provider data from the health service provider data sources. The health service provider data may include PHI and non-PHI data. The method includes storing the PHI data and the non-PHI data in an on-premises operational data store that is located on-premises of the health service provider. The method includes obtaining data analytics based on the PHI data and the non-PHI data stored in the on-premises operational data store. The method also includes communicatively coupling with a multi-tenant cloud via a global network and synchronizing the non-PHI data in the on-premises operational data store with the multi-tenant cloud via the global network.

In another example implementation, a method includes communicatively coupling with health service provider data sources. The method includes extracting health service provider data from the health service provider data sources. The health service provider data includes PHI data and non-PHI data. The method includes storing the PHI data and the non-PHI data in an on-premises operational data store. The method includes obtaining data analytics based on the PHI data and the non-PHI data in the on-premises operational data store. The method also includes generating an output record that includes the obtained data analytics results based on the PHI data and the non-PHI data in the on-premises operational data store.

Another example implementation includes an agent. The agent is located on-premises of a health service provider. The agent includes a data intake and mapping module, an on-premises operational data store, an analysis module, and a HIPAA-compliant cloud synchronization module. The data intake and mapping module is configured to interface with multiple health service provider data sources that are located on-premises of the health service provider and to extract the health service provider data that includes PHI data from the health service provider data sources. The on-premises operational data store is coupled to the data intake and mapping module. The on-premises operational data store is configured to receive at least a portion of the extracted health service provider data from the data intake and mapping module and to store the received health service provider data. The agent includes an analysis module coupled to the on-premises operational data store. The analysis module is configured to analyze the health service provider data stored on the on-premises operational data store. The agent includes a HIPAA-compliant cloud synchronization module configured to synchronize non-PHI data absent of the PHI data stored on the on-premises operational data store with a cloud-based operational data store of a health service provider tenant instance of a multi-tenant cloud off-premises of the health service provider.

This Summary introduces a selection of concepts in a simplified form that are further described below in the Detailed Description. This Summary is not intended to identify key features or essential characteristics of the claimed subject matter, nor is it intended to be used as an aid in determining the scope of the claimed subject matter.

### BRIEF DESCRIPTION OF THE DRAWINGS

Example embodiments will be described and explained with additional specificity and detail through the use of the accompanying drawings in which:

FIG. 1 is a representation of an example system for value driven outcomes in which one or more embodiments may be implemented;

FIG. 2A illustrates a representation of an example embodiment of an agent that may be implemented in the system of FIG. 1;

FIG. 2B illustrates a representation of an example embodiment of an analysis module that may be implemented in the agent of FIG. 2A;

FIG. 3A illustrates a representation of an example embodiment of a multi-tenant cloud that may be implemented in the system of FIG. 1;

FIG. 3B illustrates a representation of an example embodiment of shared services that may be implemented in the multi-tenant cloud of FIG. 3A;

FIG. 4A illustrates a representation of an example health services provider tenant instance that may be implemented in the system of FIG. 1;

FIG. 4B illustrates a representation of an example analysis module that may be implemented in the health services provider tenant instance of FIG. 4A;

FIGS. 5A-5E illustrate example reports and example dashboards that may be generated by the system of FIG. 1;

FIG. 6 is a flow chart of an example method of healthcare data management;

FIG. 7 is a flow chart of an example method of direct care cost allocation;

FIGS. 8A-8C are a flow chart of another example method of healthcare data management; and

FIG. 9 is a flow chart of another example method of healthcare data management;

FIG. 10 illustrates a representation of an example computing system that may be implemented for value driven outcomes,

all arranged in accordance with at least one embodiment described herein.

### DETAILED DESCRIPTION

Reference will be made to the drawings and specific language will be used to describe various aspects of the disclosure. Using the drawings and description in this manner should not be construed as limiting its scope. Additional aspects may be apparent in light of the disclosure, including the claims, or may be learned by practice. The drawings are non-limiting, diagrammatic, and schematic representations of example embodiments, and are not necessarily drawn to scale.

In seeking to improve care value, a challenge most healthcare delivery organizations face is limited capacity to measure and analyze healthcare value, particularly care costs. Understanding care costs is challenging due to the highly complex, fragmented, and variable nature of healthcare delivery. Billing charges are often confused with the costs of delivering care. However, charges are an inaccurate estimate of the actual costs incurred. True costing numbers may be important for developing and monitoring strategies to reduce costs, supporting the adoption of value-based reimbursement systems, and encouraging innovation. Cost accounting may also be relevant for biomedical informatics.

Some attempts have been made to address the above-mentioned issues, but the proposed solutions include barriers to adoption and other deficiencies. For example, some approaches include a lack of detailed technical implementation guidance in the literature. Many of the approaches focus on activity-based costing (costing based on detailed tracking of all activities involved in a patient's care), which may be accurate, but too resource-intensive for implementing in large healthcare organizations. Some approaches use inflexible system architectures that are difficult to customize. Certain approaches rely on frequent manual data capture, which is resource-intensive and difficult to maintain. Many approaches are not applied because of insufficient evidence that a meaningful cost-accounting system can be implemented rapidly to provide institutional benefit.

Another challenge in measuring and analyzing healthcare value is maintaining compliance with health information laws, regulations, and patient privacy standards. For example, healthcare organizations must comply with the Health Insurance Portability and Accountability Act (“HIPAA”) which regulates the use and disclosure of Protected Health Information (“PHI”). Unless a healthcare organization restricts access to PHI, the unauthorized release of a person's medical history and images may violate the patient's privacy. Accordingly, HIPAA regulations prescribe certain precautions for healthcare organizations in handling PHI data and related systems. Violations of HIPAA can result in an investigation by federal authorities and civil financial penalties. Thus, healthcare organizations may be reluctant to grant access to their electronic records and many healthcare organizations take additional precautions to avoid HIPAA violations.

Accordingly, the present disclosure generally relates to systems for value driven outcomes to understand and improve healthcare value. The systems may be rapidly implemented and iteratively enhanced to assist healthcare organizations in evaluating costs relative to outcomes to support value improvement. The systems may facilitate healthcare organizations to maintain compliance with data and/or privacy regulations and/or take additional measures to secure patient data.

For example, in some embodiments, the systems may include technology automation of various operational innovations packaged in a cloud service through a subscription model. The systems may provide initiative recommendations and tracking to drive accountability for results. The systems may facilitate in delivering information, suggesting improvement initiatives, tracking implementation of initiatives, and/or driving incremental improvements in outcomes, cost, revenue and other metrics. The systems may facilitate a reduction in an amount of professional service required for installation and configuration.

In some embodiments, the systems may facilitate efficient management of resources, workload, and throughput. The systems may be scalable, which may include the capability to linearly increase service capacity by increasing resources. The systems may be interoperable. For example, the systems may include the ability to interact efficiently with other systems and applications. The systems may increase the availability of resources to users. The systems may be maintainable including the ability to quickly and easily address defects and/or may be adaptable to changing business processes. The systems may be extensible including the ability to be extended to incorporate new business needs. The systems may facilitate management including health monitoring, configuration, deployment, and/or upgrades. The systems may facilitate security including aspects such as authentication, authorization, integrity, and/or auditing.

As used in this disclosure, “PHI data” may refer to one or more of: any information about health status, provision of health care, or payment for healthcare that can be linked to a specific individual; any data that includes any part of a patient's medical record or payment history; data that has not been anonymized or de-identified; protected information as defined by current and/or future privacy rules, laws, regulations and/or standards; protected health information as defined by HIPAA; and/or data linked to one or more identifiers as prescribed by HIPAA currently or in the future (e.g., names; geographical identifiers; specified dates; phone numbers; fax numbers; email addresses; Social Security numbers; medical record numbers; health insurance beneficiary numbers; account numbers; certificate/license numbers; vehicle identifiers and serial numbers; device identifiers and serial numbers; Web Uniform Resource Locators (URLs); Internet Protocol (IP) address numbers; biometric identifiers, including finger, retinal and voice prints; full face photographic images and any comparable images; and/or any other unique identifying number, characteristic, or code except the unique code assigned by the investigator to code the data).

In some circumstances, PHI data may be modified to transform the data into non-PHI data. For example, data may be anonymized or de-identified rendering the data non-PHI data. In some configurations, identifying information may be removed from data, resulting in non-PHI data. As used in this disclosure, “non-PHI data” may refer to one or more of: any data that does not include PHI data as described above; PHI data that has been anonymized or de-identified; and/or any data that is not linked to identifying information.

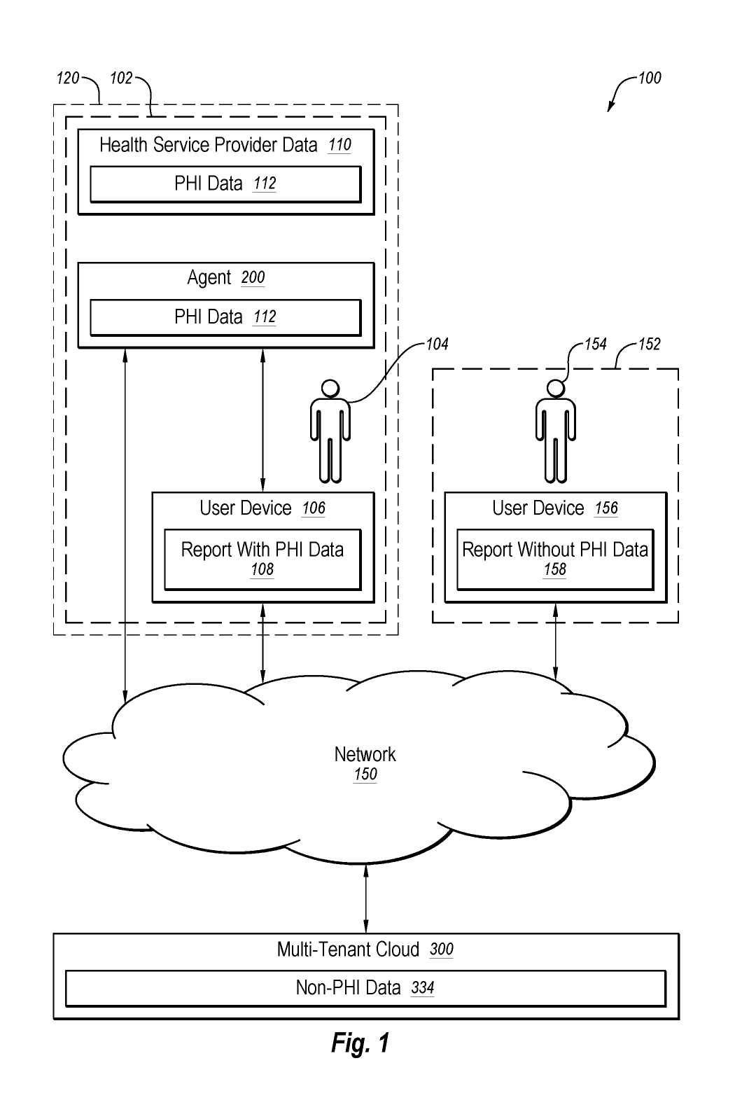

FIG. 1 is a representation of an example system 100 for value driven outcomes in which one or more embodiments may be implemented. The system 100 is generally configured to synchronize data on the premises of a health services provider (in FIG. 1, 102) with an off-premises multi-tenant cloud 300 that provides cloud-based services to the health services provider. The system 100 may be configured to maintain PHI data 112 on the premises of the health services provider and to synchronize non-PHI data 334 with the multi-tenant cloud 300. Accordingly, the system 100 may provide health services providers a specialized data management approach that enables compliance with HIPAA privacy mandates.

As illustrated, the system 100 may include an agent 200. The agent 200 may be configured to exchange data with a multi-tenant cloud 300 via a network 150. The agent 200 may be communicatively coupled to one or more sources of health service provider data (“provider data”) 110. The agent 200 may be configured to interface with the sources of provider data 110 to extract data, (e.g., electronic health records, clinical data, accounting data, etc.) regarding a health service provider and/or a patient of the health service provider. The extracted data may be processed, analyzed, organized, and/or stored by the agent 200 and/or the multi-tenant cloud 300. Some additional details of operations performed using the extracted data are provided elsewhere in the present disclosure.

As illustrated, the provider data 110 may include PHI data 112. Data stored at the agent 200 may include the PHI data 112. The multi-tenant cloud 300 may include non-PHI data 334. In FIG. 1, the PHI data 112 and the non-PHI data 334 are represented as discrete elements. In some embodiments, the PHI data 112 and/or the non-PHI data 334 may be dispersed among other data. In some embodiments, the multi-tenant cloud 300 may be absent the PHI data 112. The non-PHI data 334 may include data that does not include identifying information and portions of the PHI data 112 that has been anonymized or de-identified.

In the system 100 of FIG. 1, the provider data 110 and the agent 200 may be located on a premises 102 of the health service provider (“health service provider premises”). The premises 102 may represent a physical or geographic location of a facility of the health service provider, for instance. The premises 102, however, are not limited to a geographic location. Additionally, the premises 102 may coincide with computing systems or networks under direct or indirect control of the health service provider. For example, the premises 102 may coincide with a firewall 120. The firewall 120 may secure computing systems coupled to a network under the control of the health service provider. “On-premises 102” is used in this disclosure to describe activities or entities that exist or occur on the premises 102.

The system 100 may be configured to interact with one or more users 104 and 154. A user 104 may be on-premises 102. A user 154 may not be on the premises 102. Instead, the user 154 may be off-premises 152. “Off-premises 152” is used in this disclosure to describe activities or entities that exist or occur off the premises 102 and on the outside premises 152.

The system 100 may include user devices such as a user device 106 and a user device 156. The user devices 106 and 156 may be personal computers, hand-held devices, mobile phones, multi-processor systems, consumer electronics devices, network PCs, minicomputers, mainframe computers, laptop computers, portable electronic devices, cellular/mobile/smart phones, tablet personal computers, personal digital assistants, and/or any other suitable devices.

In some circumstances, the user device 106 may correspond to the on-premises user 104, and the user device 156 may correspond to the off-premises user 154. In other circumstances, the user devices may correspond to multiple users and/or users may have multiple corresponding devices. Furthermore, in some configurations, the user devices 106, 156 may be installed in a specific location, for example, in a location on-premises 102 or in a location off-premises 152. In other configurations, the user devices 106, 156 may be mobile and may be transported between on-premises 102 locations or off-premises 152 locations, for example, by a user.

The users 104 and 154 may include staff of the health services provider such as physicians, medical staff, administrative employees, quality managers, directors, accounting employees, information technologies (IT) staff, and/or other staff. In some circumstances, the users 104, 154 may be patients or third parties.

The system 100 may be configured to interact differently with the user devices 156 and 106 depending on whether the user devices 156 and 106 are on-premises 102 versus user devices being off-premises 152. The system 100 may be configured to detect and/or identify whether user devices 156 and 106 are on-premises 102 or off-premises 152. The system 100 may be configured such that the user devices 156 and 106 that are on-premises 102 such as the user device 106 in FIG. 1, may receive PHI data (e.g., from the agent 200, etc.). Additionally or alternatively, the system 100 may be configured such that off-premises user devices, such as the device 156, may not receive PHI data (e.g., from the agent 200, etc.).

As illustrated, the user device 106 is positioned on-premises 102. The system 100 may be configured such that the user device 106 may be communicatively coupled to the agent 200 when the user device 106 is positioned on-premises 102. Specifically, the agent 200 and/or the user device 106 may be configured to permit data transmission between one another when the user device 106 is on-premises 102. Additionally or alternatively, the system 100 may be configured such that the user device 106 may be communicatively coupled to the multi-tenant cloud 300. Specifically, the multi-tenant cloud 300 and/or the user device 106 may be configured to transmit data to and/or from the user device 106 when the user device 106 is on-premises 102. For example, the user device 106 and the multi-tenant cloud 300 may transmit data to one another via the network 150.

As illustrated, the user device 156 is positioned off-premises 152. The system 100 may be configured such that the user device 156 may not be communicatively coupled to the agent 200 when the user device 156 is positioned off-premises 152. In such configurations, the user device 156 may be communicatively coupled to the multi-tenant cloud 300, but not the agent 200.

For example, the agent 200 and/or the user device 156 may be configured to prevent data transmission between one another when the user device 156 is off-premises 152. The off-premises user device 156 may not be coupled to any devices, components, and/or portions of the system 100 positioned on-premises 102. Moreover, the off-premises user device 156 may be isolated from all devices, components, and/or portions of the system 100 on-premises 102. In some configurations, the user device 156 may only be partially isolated from devices, components, and/or portions of the system 100 positioned on-premises 102.

Additionally, the off-premises user device 156 may not receive PHI data 112 when it is positioned off-premises 152. For instance, the off-premises user device 156 may not receive PHI data 112 whether or not the user device 156 is communicatively coupled to any devices, components, and/or portions of the system 100 positioned on-premises 102. The system 100 may be configured such that the user device 156 may not receive any PHI data 112 from any devices, components, and/or portions of the system 100 positioned on-premises 102. The agent 200 may be communicatively coupled to the off-premises user device 156 and configured not to transmit PHI data to the off-premises user device 156.

The system 100 may be configured to generate reports that may be displayed to users via the user devices 106 and 156. For example, the user device 106 may display a report 108 to the user 104 and the user device 156 may display a report 158 to the user 154. As illustrated, the report 108 displayed by the user device 106 that is on-premises 102 may include the PHI data 112 and the report 158 displayed by the user device 156 that is off-premises 152 may be absent the PHI data 112.

The report 108 may be generated at the agent 200 and/or the multi-tenant cloud 300. For example, the agent 200 may generate the report 108 based at least in part on the PHI data 112 and transmit the report 108 including at least a portion of the PHI data 112 to the user device 106 to be displayed to the user 104 who is on-premises 102. In another example, the agent 200 and the multi-tenant cloud 300 may both contribute to generating the report 108 and transmitting the report 108 including at least a portion of the PHI data 112 to the user device 106 to be displayed to the user 104.

The report 108 may also be generated at the user device 106. In such configurations, the user device 106 may receive data from the agent 200 that may include the PHI data 112. Additionally or alternatively, the user device 106 may receive data from the multi-tenant cloud 300 (such data may include the non-PHI data 334 that is absent the PHI data 112). The user device 106 may generate the report 108 based at least in part on the data received from the multi-tenant cloud 300 and/or the agent 200. The user device 106 may display the generated report 108 including the PHI data 112 to the on-premises user 104.

The report 158 may be generated at the multi-tenant cloud 300. For example, the multi-tenant cloud 300 may generate the report 158 based at least in part on the non-PHI data 334 and transmit the report 158 without the PHI data 112 to the user device 156 to be displayed to the user 154 who is off-premises 152.

In some configurations, the report 158 may be generated at the user device 156. In such configurations, the user device 156 may receive data from the multi-tenant cloud 300 that may include at least a portion of the non-PHI data 334. Additionally or alternatively, the user device 156 may receive non-PHI data 334 from the agent 200 that does not include the PHI data 112. The user device 156 may generate the report 158 absent the PHI data 112 based at least in part on the data received from the multi-tenant cloud 300. The user device 156 may display the generated report 158 without PHI data 112 to the off-premises user 154.

The network 150 may be configured to communicatively couple the agent 200, the multi-tenant cloud 300, and the user devices 106, 156, to one another. The network 150 may be a global network. The network 150 may be any network or configuration of networks configured to send and receive communications between devices. In some implementations, the network 150 may include a conventional type network, a wired or wireless network, and may have numerous different configurations. Furthermore, the network 150 may include a local area network (LAN), a wide area network (WAN) (e.g., the Internet), or other interconnected data paths across which multiple devices and/or entities may communicate. In some implementations, the network 150 may include a peer-to-peer network. Additionally or alternatively, the network 150 may be coupled to or may include portions of a telecommunications network for sending data in a variety of different communication protocols. In some implementations, the network 150 includes Bluetooth® communication networks or a cellular communications network for sending and receiving communications and/or data including via short message service (SMS), multimedia messaging service (MMS), hypertext transfer protocol (HTTP), direct data connection, wireless application protocol (WAP), e-mail, etc. The network 150 may include a mobile data network that may include third-generation (3G), fourth-generation (4G), long-term evolution (LTE), long-term evolution advanced (LTE-A), Voice-over-LTE (VoLTE) or any other mobile data network or combination of mobile data networks. Furthermore, the network 150 may include one or more IEEE 802.11 wireless networks.

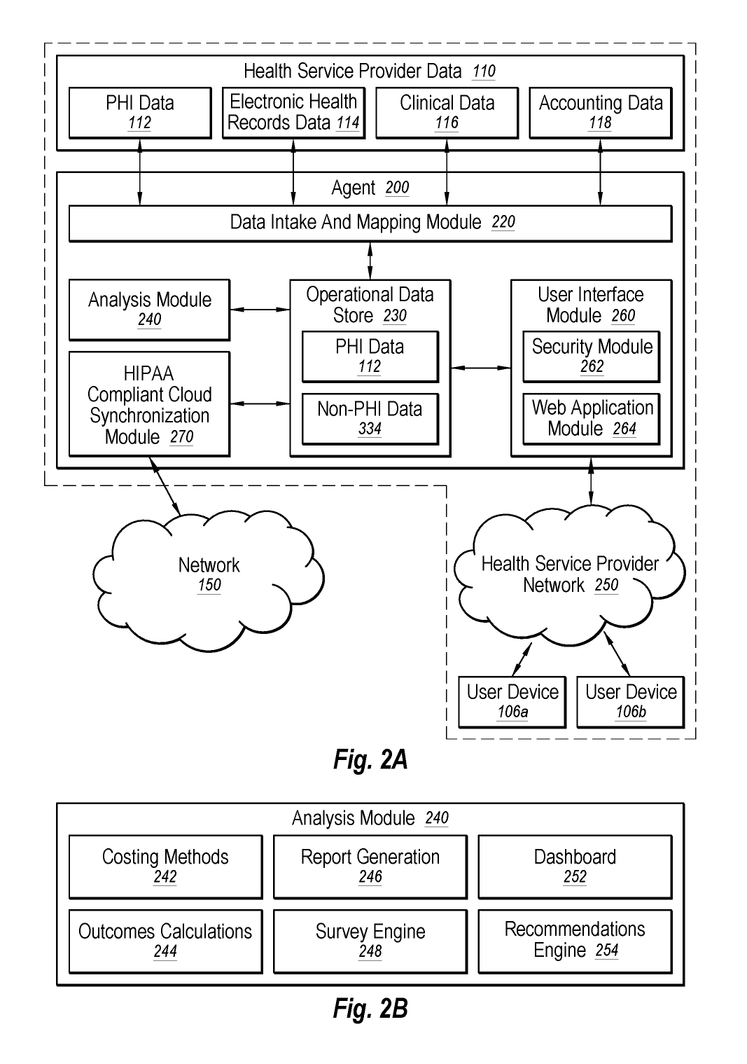

FIG. 2A illustrates a representation of an example embodiment of the agent 200 that may be implemented in the system 100 of FIG. 1. As illustrated, the provider data 110 may include electronic health records data 114, clinical data 116, accounting data 118, as well as other data. As discussed above, the provider data 110 may also include the PHI data 112. Although the PHI data 112 is represented as a discrete element, it may also be dispersed among the provider data 110, for example, in the electronic health records data 114, the clinical data 116, or other data.

The provider data 110 may be stored on one or more databases, computer systems, servers, data storages, and/or memories of the health service provider. The provider data 110 may include many different sources of data that may be positioned in different locations on-premises 102 and/or on different systems of the health service provider.

In some circumstances, different types of provider data 110 may be stored in dedicated systems. For example, the electronic health records data 114 may be stored in a dedicated health records system (e.g., databases, computer systems, servers, data storages, memories and/or etc.), the clinical data 116 may be stored in a dedicated clinical data system, the accounting data 118 may be stored in a dedicated accounting data system, and so on for different types of provider data 110. The dedicated systems may include corresponding software for organizing, storing, analyzing, accessing, displaying, and/or extracting the stored data. Examples of such software and/or systems may include Epic, Lawson, PeopleSoft Finance, PeopleSoft HR, Kronos, McKesson, CERNER, etc.

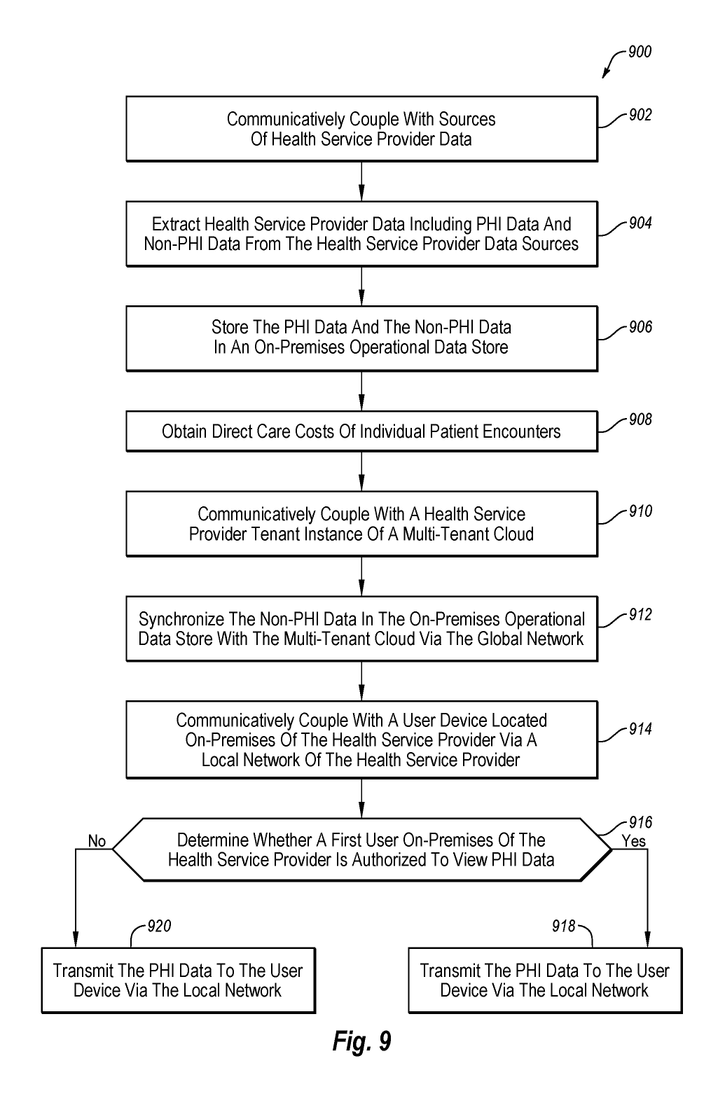

The provider data 110 may be stored in computer-readable storage media for carrying or having computer-executable instructions or data structures stored thereon. Some additional details of the computer-readable storage media are provided with reference to FIG. 9.

The provider data 110 may be communicatively coupled to the agent 200. The agent 200 may be configured to interface with one or more sources of the provider data 110. The agent 200 may be configured to extract, synchronize, analyze, process, organize, and/or store data obtained from the provider data 110 sources.

The agent 200 may include one or more computing systems of the health service provider. The agent 200 may be software installed on one or more computing systems of the health services provider. The agent 200 may be a single computing system that includes one or more processors and memory, such as a server or some other computing system or the multiple computing systems, such as multiple servers, that are networked together and configured to perform a task.

The agent 200 may include a data intake and mapping module (“mapping module”) 220 communicatively coupled to the sources of the provider data 110. The mapping module 220 may be configured to interface with the sources of the provider data 110. For example, the mapping module 220 may include instructions stored in memory that, when executed by a processor, cause the mapping module 220 to interface with the sources of the provider data 110 and/or receive data from the sources of the provider data 110. The agent 200 may be configured to receive data from the sources of the provider data 110 immediately or without material delay after the agent 200 is communicatively coupled to the sources of the provider data 110.

The mapping module 220 may include pre-loaded instructions for interfacing with the sources of the provider data 110. The pre-loaded instructions may be based on the types of interfaces between the sources of the provider data 110 and/or the mapping module 220. Additionally, the pre-loaded instructions may be based on one or more data storage formats of the provider data 110.

The mapping module 220 may be configured to execute algorithms stored in memory that cause the mapping module 220 to identify the types of interfaces between the mapping module 220 and the provider data 110 and/or one or more of the data storage formats of the provider data 110. The mapping module 220 may receive instructions from a user (e.g., 104 or 154 of FIG. 1) to configure the mapping module 220 to interface with the sources of the provider data 110.

The mapping module 220 may include machine learning algorithms (not shown), which may be stored in memory, that, when executed, may cause the mapping module 220 to interface with the sources of the provider data 110. The machine learning algorithms may cause the mapping module 220 to automatically identify the types of interfaces between the mapping module 220 and the provider data 110 and/or one or more of the data storage formats of the provider data 110. Additionally or alternatively, the machine learning algorithms may cause the mapping module 220 to configure data extraction and/or mapping processes configured to be executed by the mapping module 220.

The mapping module 220 may receive data from the sources of the provider data 110. The mapping module 220 may be configured to extract data from the sources of the provider data 110. The mapping module 220 may be configured to receive, process, analyze, and/or organize data received from the sources of the provider data 110.

In some circumstances, users may configure aspects of the mapping module 220 to provide mapping and/or data source parameters, for example. The users may configure the mapping module 220 to optimize and/or fully implement data extraction after the mapping module 220 has automatically interfaced with the sources of the provider data 110. In some configurations, the mapping module 220 may configure itself automatically.

The mapping module 220 may be configured to perform Extract, Transform and Load (“ETL”) processes. For instance, the mapping module 220 may extract data from the sources of the provider data 110 into a de-normalized abstract database, such as an operational data store (“ODS”) 230. As illustrated, the mapping module 220 may be communicatively coupled to the ODS 230.

The ODS 230 may include a database that integrates data from multiple sources and/or transmits the data to a data warehouse communicatively coupled to the ODS 230. The mapping module 220 may be configured to populate the ODS 230. For example, the mapping module 220 may organize received data and store the organized data in the ODS 230. The mapping module 220 may process data by executing algorithms stored in memory. For example, the mapping module 220 may execute cleansing, mapping, referencing and/or other suitable algorithms to process data.

The ODS 230 may include the PHI data 112 and the non-PHI data 334. The PHI data 112 and the non-PHI data 334 are represented as discrete elements in FIG. 2A, however, the PHI data 112 and non-PHI data 334 may be dispersed among one another and/or other data. The mapping module 220 may organize received data to separate the PHI data 112 from the non-PHI data 334 and/or store the non-PHI data 334 separate from the PHI data 112 in the ODS 230. The ODS 230 may include data marts for organizing data. For example, data may be organized into, clinical data marts, financial data marts, and/or other data marts within the ODS 230.

The agent 200 may include an analysis module 240. The analysis module 240 may be configured to analyze data in the ODS 230. The analysis module 240 may receive data from the ODS 230, for example the PHI data 112 and/or the non-PHI data 334. The analysis module 240 may include algorithms stored in memory to perform calculations and/or operations on the PHI data 112 and/or the non-PHI data 334. Additionally or alternatively, the analysis module 240 may be configured to execute algorithms stored in memory to perform calculations and/or operations on the PHI data 112 and/or the non-PHI data 334. The analysis module 240 may apply algorithms to analyze the data in the ODS 230 to, for example: identify general ledger costs attributable to direct patient care; allocate costs of individual patient encounters based on modular costing business rules; calculate patient-level quality and/or outcome metrics; and/or provide other services as discussed in further detail below.

The agent 200 may also include a user interface module 260. The user interface module 260 may be configured to be communicatively coupled with user devices 106a and 106b via a health service provider network 250. The user interface module 260 may be communicatively coupled to the ODS 230 to permit users to access data.

The user interface module 260 may include a security module 262 and a web application module 264. The security module 262 may be configured to control and/or regulate access to data (e.g., the PHI data 112, the non-PHI data 334, etc.). Additionally, the security module 262 may be configured to secure data and/or facilitate compliance with data regulations such as HIPAA. The web application module 264 may be configured to generate and/or host an application such as a web application for the user devices 106a, 106b. The web application module 264 may permit users to interact with the agent 200, for example, to access data in the ODS 230 such as the PHI data 112 and/or the non-PHI data 334. Additionally or alternatively, the web application module 264 may be configured to transmit and/or generate reports for displaying to users by the user devices 106a, 106b (see, for example, the report 108 in FIG. 1 and associated description).

In some embodiments, the health service provider network 250 may be a local network, rather than a global network or some implementation of the network 150. Although the health service provider network 250 is represented as a discrete element in FIG. 2A, the health service provider network 250 may encompass all or some network connected features located on-premises 102. For example, in some aspects the provider data 110, the agent 200, the user devices 106a, 106b and/or other features may be part of the health service provider network 250, which is substantially similar to the network 150 described with reference to FIG. 1.

The security module 262 may facilitate the health service provider in complying with applicable HIPAA regulations, for example, by securing the PHI data 112 and/or controlling access to the PHI data 112. In some implementations, the security module 262 may be configured to facilitate the health service provider in complying with all or substantially all applicable HIPAA regulations. Additionally or alternatively, the security module 262 may be configured to comply with all or substantially all applicable HIPAA regulations. Additionally or alternatively, the security module 262 may be configured to include data security features, protocols, and/or programs that go beyond the requirements of HIPAA regulations. For the purposes of this disclosure, such additional security features, protocols, and/or programs may be referred to as “additional precautions.”

The security module 262 may be configured to control and/or monitor electronic access to the PHI data 112. The security module 262 may enforce precautions such as access rules for the PHI data 112 and related system features. The security module 262 may be configured to permit certain users to access the PHI data 112 and/or prohibit other users from accessing the PHI data 112. For example, the security module 262 may identify users and grant access to users based on identifiers (usernames, reference numbers, passkeys, passwords, globally unique identifier, etc.). Users may input the identifiers, for example, at the user devices 106a and/or 106b. The users may input identifiers via web applications displayed on one or more of the user devices 106a and/or 106b. The security module 262 may include any suitable security features, protocols, and/or programs. In other configurations, the security module 262 may limit access to the PHI data 112 to properly authorized individuals.

The security module 262 may be configured to authenticate any entity that accesses and/or attempts to access the PHI data 112. Additionally, the security module 262 may be configured to authenticate any entity that communicates and/or attempts to communicate PHI data 112 with the health services provider. Authentication may include use of corroborating password systems, two or three-way handshakes, telephone callback procedures, token systems, etc. The security module 262 may also be configured to control alteration and/or modification of the PHI data 112 by unauthorized individuals and/or in an unauthorized manner.

The security module 262 may control and/or monitor physical access to components and/or systems housing PHI data 112. For example, the security module 262 may limit access to locations where components and/or systems (e.g., servers, computer systems, databases, etc.) that store the PHI data 112 are located. The security module 262 may include features to limit physical access to such components and/or systems to properly authorized individuals. For example, the security module 262 may control an electronic lock on a door of a room housing the components and/or systems including the PHI data 112. Additionally or alternatively, the system 100 may include other physical and/or electronic security features to protect PHI data 112 and/or comply with applicable HIPAA regulations.

The agent 200 may also include a HIPAA compliant cloud synchronization module (“synchronization module”) 270 communicatively coupling the agent 200 with the network 150. The synchronization module 270 may be configured to communicatively couple the agent 200 to the multi-tenant cloud 300 via the network 150. The synchronization module 270 may include algorithms stored in memory that may be executed by a processor to synchronize (or “sync”) the agent 200 with the multi-tenant cloud 300.

The synchronization module 270 may facilitate the health service provider in complying with applicable HIPAA regulations, for example, by securing PHI data 112 and/or controlling access to PHI data 112. In some implementations, the synchronization module 270 may be configured to facilitate the health service provider in complying with all (or substantially all) applicable HIPAA regulations. Additionally or alternatively, the synchronization module 270 itself may be configured to comply with all (or substantially all) applicable HIPAA regulations. Additionally or alternatively, the synchronization module 270 may be configured to include additional precautions such as data security features, protocols, and/or programs that go beyond the requirements of HIPAA regulations.

The synchronization module 270 may synchronize data in the ODS 230 with corresponding data storage in the multi-tenant cloud 300. Synchronizing data may include updating data stored at the multi-tenant cloud 300 with data stored at the agent 200, vice versa, updating data at the multi-tenant cloud 300 and/or the agent 200 based on certain rules, and transmitting data from the ODS 230 to the multi-tenant cloud 300 via the network 150, or some combination thereof. The synchronizing data may refer to one-way file synchronization or two-way file synchronization. In some configurations, synchronizing data may include automatically copying data that needs to be updated and/or preventing copying of data that is already synchronized.

In some configurations, the synchronization module 270 may be configured to synchronize only non-PHI data 334 with the multi-tenant cloud 300. In this and other configurations, the synchronization module 270 may detect and/or identify whether data is PHI data 112 or non-PHI data 334. The synchronization module 270 may separate non-PHI data 334 from PHI data 112. The synchronization module 270 may then transmit only the non-PHI data 334 to the multi-tenant cloud 300 via the network 150. Additionally, the synchronization module 270 may be configured to anonymize and/or de-identify PHI data 112, rendering it non-PHI data 334, before transmitting the non-PHI data 334 to the multi-tenant cloud 300 via the network 150.

In some implementations, the synchronization module 270 may be configured to encrypt and/or compress data before it is transmitted to the multi-tenant cloud 300 via the network 150 and to detect conflicting and/or inconsistent data and/or data modifications.

FIG. 2B illustrates a representation of an example embodiment of the analysis module 240 that may be implemented in the agent 200 of FIG. 2A. The analysis module 240 may be configured to provide services such as data analytics, costing methods 242, outcomes calculations 244, report generation 246, survey engine 248, dashboard 252, recommendations engine 254 and/or other suitable services. In other configurations, any services described with respect to the analysis module 240 may be included and/or provided by other portions of the agent 200. For example, one or more of the data analytics, the costing methods 242, the outcomes calculations 244, the report generation 246, the survey engine 248, the dashboard 252, the recommendations engine 254 and/or other services may be included or provided as part of the user interface module 260.

The costing methods 242 may include executing costing methods 242 to perform operations and/or calculations on the PHI data 112 and/or the non-PHI data 334, as described in further detail below. The costing methods 242 may include cost mapping. For example, the costing methods 242 may include one or more mapping tables and the analysis module 240 may map data based on the mapping tables. The one or more mapping tables and/or the costing methods 242 may be configurable and/or modifiable by a user.

The outcomes calculations 244 may include executing outcomes algorithms to perform operations and/or calculations on the PHI data 112 and/or the non-PHI data 334 to obtain outcome information, as described in further detail below. The outcomes calculations 244 may be configurable and/or modifiable by a user. The outcomes calculations 244 may be configured to facilitate evaluating patient care quality, outcomes, and/or value measurements. Additionally, the outcomes calculations 244 may be configured to correlate quality, outcomes, and/or value measurements to the costs obtained by the costing methods 242.

The report generation 246 may include executing algorithms to perform operations and/or calculations on the PHI data 112 and/or the non-PHI data 334 to generate reports to be transmitted to user devices and/or users, such as the user device 106 and/or the report 108 of FIG. 1. The report generation 246 may perform operations and/or calculations on at least a portion of the PHI data 112 and/or the non-PHI data 334, and generate a report customized for a user or based on input from a user. The report generation 246 may be configurable and/or modifiable by a user or automatically configuring based on any suitable circumstances such as the user requesting the report, the data requested, and/or other circumstances. The report generation 246 may include department-specific reports for the health services provider to provide a customized experience for users in specific departments. Additionally, department-specific reports may optimize performance by limiting datasets.

The survey engine 248 may include executing algorithms to generate surveys. In some configurations, surveys may be based on user input, the PHI data 112, the non-PHI data 334, or some combination thereof. The survey engine 248 may be configurable and/or modifiable by a user. The survey engine 248 may automatically generate surveys based on the PHI data 112 and/or the non-PHI data 334. In some configurations, information generated by the survey engine 248 may be included in the reports provided in the report generation 246.

The survey engine 248 may be configured to receive data regarding potential and/or selected participants, for example, from the ODS 230. The survey engine 248 may be configured to identify, query, and extract data regarding potential and/or selected participants. The survey engine 248 may include forms to be used by users in formulating surveys. The survey engine 248 may receive input from users to configure surveys. The survey engine 248 may generate computerized adaptive tests (CATs).

The dashboard 252 may include executing algorithms to generate and/or host information to be displayed to users, for example, via user devices. The dashboard 252 may be a web application and/or web page generated and/or hosted by the analysis module 240 and/or displayed on a user device. The dashboard 252 may be configurable and/or modifiable by a user. Additionally or alternatively, the dashboard 252 may automatically be configured, for example, by the analysis module 240 based on any suitable circumstances such as the user accessing the dashboard, the data requested, and/or other circumstances.

The recommendations engine 254 may include executing algorithms to generate recommendations based on the PHI data 112, the non-PHI data 334 and/or data inputted from a user. The recommendations engine 254 may be configurable and/or modifiable by a user. The recommendations engine 254 may automatically generate survey data based on the PHI data 112, the non-PHI data 334 or data inputted from a user. In some configurations, information generated by the recommendations engine 254 may be included in the reports provided in the report generation 246. The report generation 246 and/or the dashboard 252 may provide and/or display information to users, for example, from the recommendations engine 254, the outcomes calculations 244, and/or the survey engine 248.

In some configurations, data obtained and/or generated by some of the services of the analysis module 240 may be used in other services provided by the analysis module 240. For example, data generated at the data analytics, the costing methods 242 and/or the outcomes calculations 244 may be used in the other services provided by the analysis module 240 such as the report generation 246 and/or the dashboard 252.

Report generation 246 and/or the dashboard 252 may be configured to provide and/or display data to users, for example, via the user interface module 260. In some circumstances, report generation 246 and/or the dashboard 252 may be part of a reporting layer configured to provide actionable information to users. Report generation 246 and/or the dashboard 252 may be configured to generate web applications including data generated by the analysis module 240 to be provided and/or displayed to users via user devices. For example, report generation 246 and/or the dashboard 252 may be configured to generate and/or host web applications including data from the data analytics, the costing methods 242 and/or the outcomes calculations 244. Additionally or alternatively, report generation 246 and/or the dashboard 252 may visualize data in various ways to convey data to users via user devices.

The report generation 246 and/or the dashboard 252 may generate and/or display web-based summaries enabling users to efficiently engage with and analyze data, for example, from the ODS 230. The report generation 246 and/or the dashboard 252 may generate and/or display web-based reports summarizing data for the health services provider corresponding to the agent 200. The summaries generated at the report generation 246/or the dashboard 252 may include dropdown menus and selectable filters. The summaries may include the ability to break a body of information down into smaller parts and/or to obtain more detailed information with a specific focus. The summaries may include hover-over and/or drill-down capabilities. Such features of the summaries may facilitate evaluation of data.

The agent 200 may include a web-based administrative console (not shown). The web-based administrative console may be part of the user interface module 260 or the analysis module 240, for instance. The web-based administrative console may provide users with the ability to customize and/or configure aspects of the agent 200. The web-based administrative console may permit users such as IT staff of the health services provider to customize and/or configure aspects of the agent 200. For example, the web-based administrative console may permit users to specify mapping parameters and/or configurations to fine-tune data flow, populate missing fields, map proprietary coding, etc. The web-based administrative console may also permit mapping parameters and/or configurations to be specified, for example, for the mapping module 220.

The web-based administrative console may further provide users with the ability to perform validation and/or supplementary tasks for the agent 200. For example, the web-based administrative console may permit users to supply user-level missing data, verify the accuracy of data, verify mapping configurations, map different uses of data at a user-level, map costing methods at a user-level, input proprietary settings for a specific health services provider installation, and so forth.

The web-based administrative console may provide the ability for cost mapping to be performed at the agent 200. For example, professional cost mapping may be performed through the web-based administrative console. Cost mapping details may be entered through the web-based administrative console. In some aspects, cost mapping may specify mapping, parameters, and/or data for labor expenses, general expenses, equipment expenses, depreciation, equipment maintenance, repair expenses, fixed expenses, direct expenses, and/or other expenses.

The web-based administrative console may provide users with the ability to enter additional data. For example, in some configurations costing data may be input via the web-based administrative console. Data such as physician salary data monthly, staff salary data, care provider identity, and/or staff identity may be input by users via the web-based administrative console.

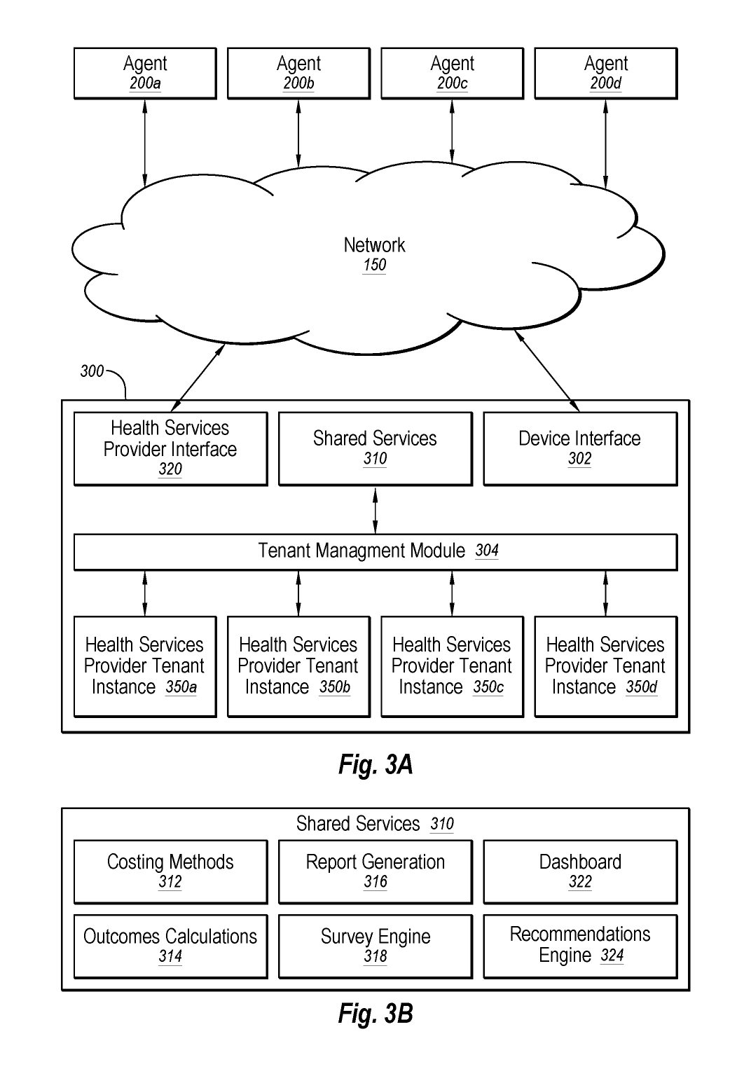

FIG. 3A illustrates a representation of an example embodiment of the multi-tenant cloud 300 that may be implemented in the system 100 of FIG. 1. The multi-tenant cloud 300 may include a load-balanced elastic cloud architecture and a multi-tenant data warehouse. The multi-tenant cloud 300 may be configured to provision health services provider tenants with health service provider tenant instances 350a-350d (generally, tenant instance 350 or tenant instances 350). Each the tenant instances 350a-d may correspond to one or more agents 200a-d of health service provider tenants. The multi-tenant cloud 300 may be a load balanced multi-tenant cloud, as described elsewhere in this disclosure. The tenant instances 350 may be virtual machines such as virtual private servers, system virtual machines, process virtual machines, etc.

In some implementations, the tenant instances 350 may be managed by a tenant management module 304. In some aspects, the tenant management module 304 may be configured to provide load balancing for the multi-tenant cloud 300 and/or generate the tenant instances 350. The tenant management module 304 may include a virtual database manager.

As illustrated, in some configurations the multi-tenant cloud 300 may include a health services provider interface 320 communicatively coupling the multi-tenant cloud 300 with the network 150. The health services provider interface 320 may be configured to communicatively couple the multi-tenant cloud 300 to multiple health services providers via the network 150. Specifically, the health services provider interface 320 may couple the multi-tenant cloud 300 to one or more agents, each corresponding to one of the health services providers, such as the agent 200 of FIGS. 1 and 2A. The health services provider interface 320 may permit data to be exchanged between the agents and the multi-tenant cloud 300 via the network 150. The health services provider interface 320 may include algorithms stored in memory that may be executed by a processor to synchronize the multi-tenant cloud 300 with the agents of the health services provider, or vice versa.

Additionally or alternatively, the multi-tenant cloud 300 may include a device interface 302 communicatively coupling the multi-tenant cloud 300 with the network 150. The device interface 302 may be configured to communicatively couple the multi-tenant cloud 300 to multiple user devices of the health services providers via the network 150. Specifically, the device interface 302 may couple the multi-tenant cloud 300 to one or more user devices, which may correspond to any of the health services providers. For example, one of the health service providers may correspond to the premises 102, the on-premises user device 106, and/or the off-premises user device 156 of FIG. 1. In such aspects, the device interface 302 may couple the multi-tenant cloud 300 to the on-premises user device 106, and/or the off-premises user device 156, via the network 150 (see for example, FIG. 1). The device interface 302 may permit data to be exchanged between the user devices of the health services providers and the multi-tenant cloud 300 via the network 150. The functionality of the device interface 302 and the health services provider interface 320 may be combined in a single interface.

The multi-tenant cloud 300 may be configured to provide shared services 310 to the tenant instances 350. FIG. 3B illustrates a representation of an example embodiment of the shared services 310 that may be implemented in the multi-tenant cloud 300 of FIG. 3A. The shared services 310 may include data analytics, costing methods 312, outcomes calculations 314, report generation 316, survey engine 318, dashboard 322, recommendations engine 324 and/or other suitable services. Any of the shared services 310 may include standard object frameworks established and optimized for the multi-tenant cloud 300 of FIG. 3A. The shared services 310 may be load-balanced services, employing load balanced resources of the multi-tenant cloud 300 to provide services for the tenant instances 350. The shared services 310 may share common resources of the multi-tenant cloud 300 for persistent access to resources and/or other functionality, as described elsewhere in this disclosure.

The agent 200 and/or the tenant instance 350 may not include some of the services provided by the analysis modules 240, 340, and such functionality may be provided solely by the shared services 310. For example, in some configurations, the agent 200 and/or the tenant instance 350 may not include survey engines 248, 328, and the multi-tenant cloud 300 may provide such functionality at the survey engine 318.

The shared services 310 may include any suitable characteristics of the services provided by the analysis module 240 described with respect to FIGS. 2A-2B. However, in some configurations, while the analysis module 240 may provide services based on both PHI data 112 and non-PHI data 334, the shared services 310 may be provided based only on non-PHI data 334. For example, in FIG. 1, the multi-tenant cloud 300 includes only non-PHI data 334 and no PHI data 112. Accordingly, in such configurations, the shared services 310 may be provided based only on non-PHI data 334 stored at the multi-tenant cloud 300. Such configurations may facilitate compliance with HIPAA regulations and/or facilitate in providing additional precautions for compliance. In other configurations, the shared services 310 may include additional services that do not include any of the services described with respect to the analysis module 240.

In some configurations, one or more of the shared services 310 (e.g., 312, 314, 316, 318, 322, 324, etc.) may be a standard object framework established and optimized for the multi-tenant cloud 300. The shared services 310 may be provided by common facilities for persistent access to the shared services 310 by the tenant instances 350, and/or other necessary functionality.

The multi-tenant cloud 300 may be configured to service various environments such as development, testing, staging, and production. The multi-tenant cloud 300 may be configured to interface with third parties, for example, via the network 150. In such configurations, the multi-tenant cloud 300 may be communicatively coupled to receive information from third parties. Information from the third parties may be used to facilitate mapping and/or processing data, either at the agent 200 and/or at the multi-tenant cloud 300 of FIG. 1.

In some configurations, any of the shared services 310 may be a stand-alone, cloud delivered tool. For example, the survey engine 318 may be a stand-alone, cloud delivered survey tool, leveraging content stored in the multi-tenant cloud 300 and/or the other shared services 310. The survey engine 318 may facilitate data collection from the agent 200, the multi-tenant cloud 300 and third parties to be used in generating the surveys.

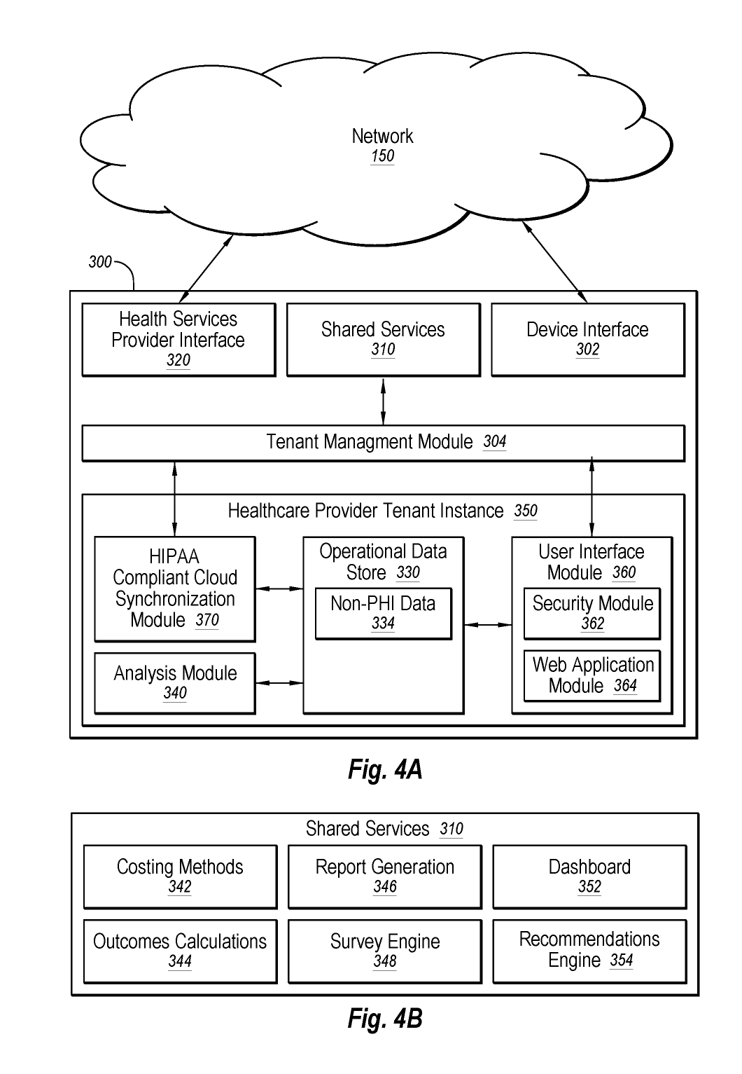

FIG. 4A illustrates a representation of an example of a health services provider tenant instance (tenant instance) 350 that may be implemented in the system 100 of FIG. 1. The tenant instance 350 may be an example of one of the tenant instances 350 of FIG. 3A. Any of the tenant instances 350 may include suitable aspects of the tenant instance 350. Additionally or alternatively, each of the tenant instances 350 may or may not be the same as one another. In some implementations, each of the tenant instances 350 may include different configurations corresponding to each different health service provider.

The tenant instance 350 may be a cloud-synchronized instance corresponding to the agent 200. The tenant instance 350 may include aspects and/or components corresponding to the agent 200 of the corresponding health service provider. Specifically, the tenant instance 350 may include an ODS 330, an analysis module 340, a user interface module 360, and/or a HIPAA compliant cloud synchronization module 370. In some implementations, the ODS 330, the analysis module 340, the user interface module 360, and/or the HIPAA compliant cloud synchronization module 370 may include any or all suitable aspects as described with respect to corresponding components of the agent 200 (e.g., ODS 230, the analysis module 240, the user interface module 260, and/or the synchronization module 270). For example, as illustrated, the user interface module 360 may include a security module 362 and/or a web application module 364 including similar or the same features as described with respect to the security module 262 and/or the web application module 264 of the agent 200.

The software, algorithms, and/or the computer readable instructions of the tenant instance 350 may be the similar or identical to those of the agent 200. In such configurations, the software, algorithms, and/or the computer readable instructions may be configured to detect whether it is loaded, positioned and/or operating on-premises 102, off-premises 152, and/or in the multi-tenant cloud 300. In some implementations, the software, algorithms, and/or the computer readable instructions may configure its operation based on the whether it is operated on-premises 102, off-premises 152, and/or in the multi-tenant cloud 300. Additionally or alternatively, in some implementations, the software, algorithms, and/or the computer readable instructions may be configured to detect which of the tenant instances 350 (described with respect to FIG. 3A) it is operating on and/or corresponds with. In such implementations, the software, algorithms, and/or the computer readable instructions may configure its operation based on which of the tenant instances it is operating on and/or corresponds with.

For example, the software, algorithms, and/or the computer readable instructions may activate and/or deactivate various components, interfaces and/or other portions in response to determining that it is being operated on-premises 102, off-premises 152, and/or in the multi-tenant cloud 300. Additionally or alternatively, the software, algorithms, and/or the computer readable instructions may configure the operation of various components, interfaces and/or other portions in response to determining that it is being operated on-premises 102, off-premises 152, and/or in the multi-tenant cloud 300.

As illustrated for example in FIG. 4A, in some configurations the tenant instance 350 may not include a component corresponding to the mapping module 220. The tenant instance 350 may be configured in such a manner, for example, because it is not directly communicatively coupled to the provider data 110 (see FIG. 2A and associated description above) and/or because it is not configured to interface and/or extract data from sources of the provider data 110. In other configurations, for example, if the tenant instance 350 is communicatively coupled to the provider data 110, the tenant instance 350 may include a component corresponding to the mapping module 220 of the agent 200. In some configurations, software, algorithms, and/or the computer readable instructions may be configured to detect whether it is coupled to the provider data 110 and/or deactivate a component corresponding to the mapping module 220 upon determining that it is not coupled to the provider data 110.

Although, as mentioned above, the ODS 330 of the tenant instance 350 may be substantially the same or similar to the ODS 230 of the agent 200, the ODS 330 may be absent the PHI data 112 and may include only the non-PHI data 334, as illustrated in the example implementation of FIG. 4A. The ODS 330 may include data marts for organizing data, such as the non-PHI data 334. For example, the non-PHI data 334 may be organized into, clinical data marts, financial data marts, and/or other data marts within the ODS 330.

The HIPAA compliant cloud synchronization module 370 may be configured to communicatively couple the tenant instance 350 to the network 150. The synchronization module 370 may be configured to communicatively couple the tenant instance 350 to the agent 200 via the network 150. As illustrated, in some configurations the synchronization module 370 may couple the tenant instance 350 to the network 150 via, for example, the tenant management module 304 and/or the health services provider interface 320. The synchronization module 370 may include algorithms stored in memory that may be executed by a processor to synchronize the tenant instance 350 with the corresponding agent 200 located on-premises 102 of the health service provider.

In some implementations, the synchronization module 370 may be the same software, algorithms, and/or the computer readable instructions as the synchronization module 270 of the agent 200. In such configurations, the software, algorithms, and/or the computer readable instructions may be configured to detect whether it is being operated on-premises 102, off-premises 152, and/or at the tenant instance 350. Furthermore, in such configurations the software, algorithms, and/or the computer readable instructions may configure itself based on whether it is being operated on-premises 102, off-premises 152, and/or at the tenant instance 350.

In some implementations, the synchronization module 370 may facilitate compliance with applicable HIPAA regulations, for example, by ensuring the tenant instance 350 is absent the PHI data 112. Additionally or alternatively, the synchronization module 370 may facilitate controlling access to PHI data 112. In some implementations, the synchronization module 370 may be configured to facilitate the health service provider operating the agent 200 in complying with all (or substantially all) applicable HIPAA regulations. Additionally or alternatively, the synchronization module 370 itself may be configured to comply with all (or substantially all) applicable HIPAA regulations. Additionally or alternatively, the synchronization module 370 may be configured to include additional precautions such as data security features, protocols, and/or programs that go beyond the requirements of HIPAA regulations.

The synchronization module 370 may synchronize data in the ODS 330 with corresponding ODS 230 of the agent 200. Synchronizing data may include updating data stored at the agent 200 with data stored at the tenant instance 350, and/or vice versa. Synchronizing data may include updating data at the tenant instance 350 and/or the agent 200 based on certain rules. Synchronizing data may include the synchronization module 370 transmitting data from the ODS 330 to the agent 200 via the network 150. In some implementations, synchronizing data may refer to one-way file synchronization or two-way file synchronization. In some configurations, synchronizing data may include automatically copying data that needs to be updated and/or preventing copying of data that is already synchronized.

In one example configuration, the synchronization module 370 may be configured to synchronize only non-PHI data 334 with the agent 200. In such configurations, the synchronization module 370 may detect and/or identify whether data is PHI data 112 or non-PHI data 334. The synchronization module 370 may receive data and/or identify whether data is non-PHI data 334 or PHI data 112. The synchronization module 370 may be configured to refuse any PHI data 112 transmitted to it from the agent 200 and/or generally via the network 150. Additionally or alternatively, the synchronization module 370 may be configured not to store any PHI data 112 in the ODS 330 and/or only store non-PHI data 334 in the ODS 330.

Additionally or alternatively, the synchronization module 370 may be configured to anonymize and/or de-identify PHI data 112, rendering it non-PHI data 334, before storing it in the ODS 330. In some implementations, the synchronization module 370 may be configured to encrypt and/or compress data before it is transmitted to the agent 200 via the network 150. In further implementations, the synchronization module 370 may be configured to detect conflicting and/or inconsistent data and/or data modifications.

As illustrated, the user interface module 360 may be configured to be communicatively coupled with the network 150, for example, via the tenant management module 304 and/or the device interface 302. The user interface module 360 may be configured to couple the tenant instance 350 with user devices 106 and/or 156 via the network 150 (see for example FIG. 1 and associated description above). The user interface module 360 may be communicatively coupled to the ODS 330 to permit users 104 and/or 154 to access data.

The security module 362 may be configured to control and/or regulate access to data (e.g., the non-PHI data 334, etc.). Additionally or alternatively, the security module 362 may be configured to secure data and/or facilitate compliance with data regulations such as HIPAA. The web application module 364 may be configured to generate and/or host an application such as a web application for the user devices 106 and/or 156. The web application module 364 may permit users 104 and/or 154 to interact with the tenant instance 350, for example, to access data in the ODS 330 such as the non-PHI data 334. Additionally or alternatively, the web application module 364 may be configured to transmit and/or generate reports for displaying to users by the user devices 106 and/or 156, such as the reports 108 and/or 158 described with respect to FIG. 1.

In some implementations, the security module 362 may facilitate the health service provider in complying with applicable HIPAA regulations, for example, by securing PHI data 112 and/or controlling access to PHI data 112. In some implementations, the security module 362 may be configured to facilitate the health service provider in complying with all (or substantially all) applicable HIPAA regulations. Additionally or alternatively, the security module 362 itself may be configured to comply with all (or substantially all) applicable HIPAA regulations. Additionally or alternatively, the security module 362 may be configured to include additional precautions such as data security features, protocols, and/or programs that go beyond the requirements of HIPAA regulations.

The security module 362 may enforce precautions such as access rules for the PHI data 112 and related system features. The security module 362 may be configured to permit certain users to access the PHI data 112 and/or prohibit other users from accessing the PHI data 112. For example, the security module 362 may identify whether the users are located on-premises 102 or off-premises 152. Additionally or alternatively, the security module 362 may grant or prohibit access to data based on the location of users.

Additionally or alternatively, the security module 362 may identify users and grant access to users based on identifiers (usernames, reference numbers, passkeys, passwords, globally unique identifier, etc.). Users may input the identifiers, for example, at the user devices 106 and/or 156. The users may input identifiers via web applications displayed on one or more of the user devices 106 and/or 156. In other configurations, the security module 362 may include any suitable security features, protocols, and/or programs. In other configurations, the security module 362 may limit access to data to properly authorized individuals.

In some implementations, the security module 362 may be configured to authenticate any entity that accesses and/or attempts to access data. Additionally or alternatively, the security module 362 may be configured to authenticate any entity that communicates and/or attempts to communicate data with the tenant instance 350. Authentication may include, but is not limited to, use of corroborating password systems, two or three-way handshakes, telephone callback procedures, token systems, etc. Additionally or alternatively, the security module 362 may also be configured to control alteration and/or modification of data at the ODS 330 by unauthorized individuals and/or in an unauthorized manner.

FIG. 4B illustrates a representation of an example analysis module 340 that may be implemented in the tenant instance 350. The analysis module 340 may include services or portions of services configured and/or tailored for individual tenant instances 350 such as the tenant instance 350, while the shared services 310 of FIG. 3A may include services or portions of services universally or generally applicable to all of the tenant instances 350.

As illustrated, in some configurations, the analysis module 340 may include data analytics, costing methods 342, outcomes calculations 344, report generation 346, survey engine 348, dashboard 352, recommendations engine 354 and/or other suitable services. The analysis module 340 may include any suitable characteristics of the services provided by the analysis module 240 as illustrated and described with respect to FIGS. 2A-2B. However, in some configurations, while the analysis module 240 may provide services based on both the PHI data 112 and the non-PHI data 334, the analysis module 340 services may be provided based only on the non-PHI data 334.

For example, with combined reference to FIGS. 1 and 3B, the multi-tenant cloud 300 includes only the non-PHI data 334 and none of the PHI data 112. Accordingly, in configurations like that of FIG. 1, the analysis module 340 may be provided based only on the non-PHI data 334 stored at the tenant instance 350. Such configurations may facilitate compliance with HIPAA regulations and/or facilitate in providing additional precautions for compliance. In other configurations, the analysis module 340 may include additional services that do not include any of the services described with respect to the analysis module 240 and/or the shared services 310.

As described above, the system 100 may include features to facilitate HIPAA compliance and, in some configurations, additional precautions for patient privacy and/or data security. Accordingly, the system 100 may decrease security risks and/or data privacy concerns. The system 100 may specifically address risks and concerns prevalent in the healthcare industry, for example, for health service providers.

The system 100 may separate and/or secure data concerning regulations such as HIPAA. The system 100 may separate the PHI data 112 from non-PHI data 334 to be used in analytics. In further configurations, the system 100 may maintain the health care service provider's PHI-data on the premises of the health service provider. Additionally or alternatively, the system 100 may maintain the health care service provider's PHI-data within the health care service provider's local, secured network. Additionally or alternatively, the system 100 may maintain the health care service provider's PHI-data behind the health care service provider's firewall. In some configurations, non-PHI data may be the only data that leaves the premises, the local/secured network, and/or the firewall secured portion of the health care service provider's systems. For example, non-PHI data (such as summary data) may be the only data that is synchronized with cloud-based components such as the multi-tenant cloud 300.

The system 100 may facilitate cloud-based services and/or distribution of data analytics for health services providers. In some aspects, the system 100 may facilitate a health services provider in quickly installing and/or activating software to begin realizing value in a fraction of the time when compared to previous systems. The system 100 may be provided to health services providers as a subscription-based service. The system 100 may be provided to health services providers as a software as a service (“SaaS”).

In some configurations, all data transmitted through the network 150 may be encrypted for security. For example, data may be encrypted and/or decrypted at the agent 200, the multi-tenant cloud 300 and/or any user devices (e.g., the user devices 106, 156, etc.). Additionally or alternatively, all data transmitted through the health service provider network 250 may be encrypted for security. In further configurations, all data transmitted as part of system 100 may be encrypted for security, whether the data is transmitted through the network 150 or other channels.

As discussed above, the system 100 may be configured to generate reports and/or dashboards to provide and/or display information to users. For example, as illustrated in FIG. 1, the report 108 may be displayed to the user 104 via the user device 106 and/or the report 158 may be displayed to the user 154 via the user device 156.

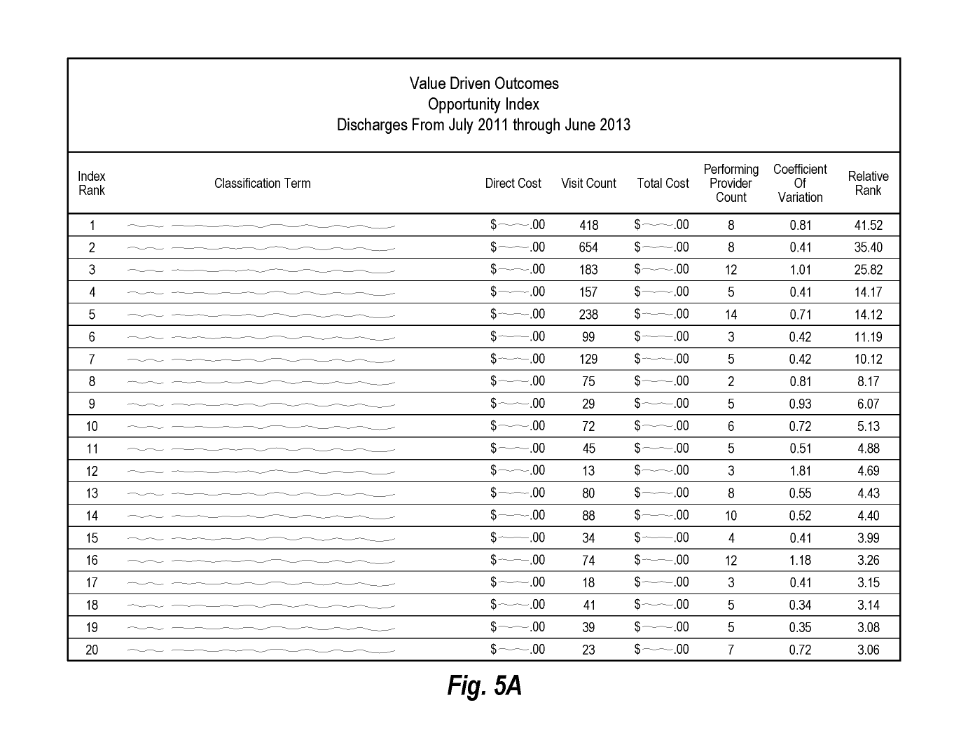

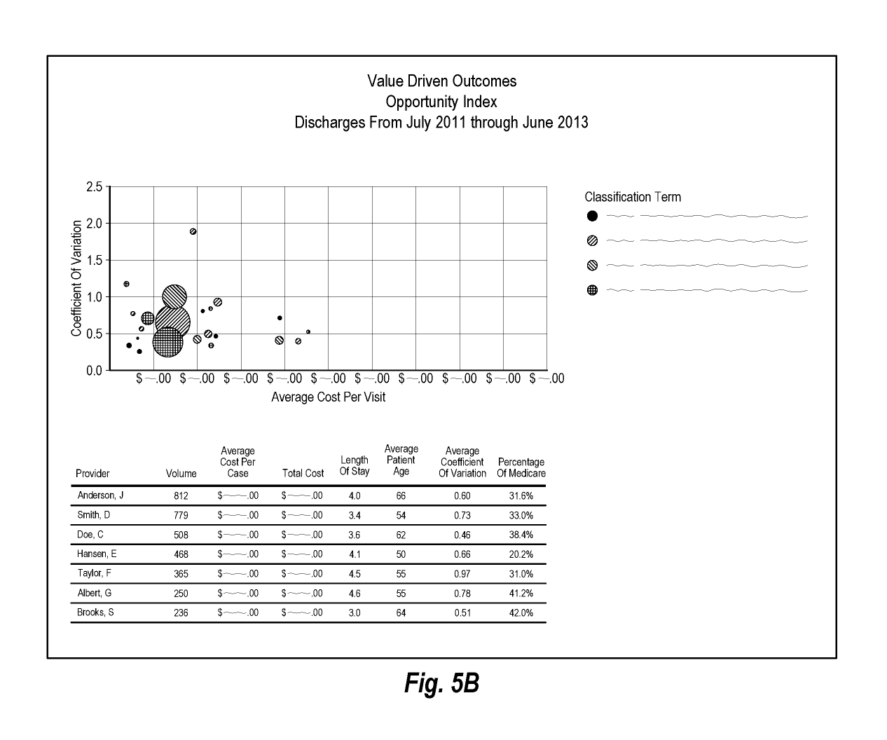

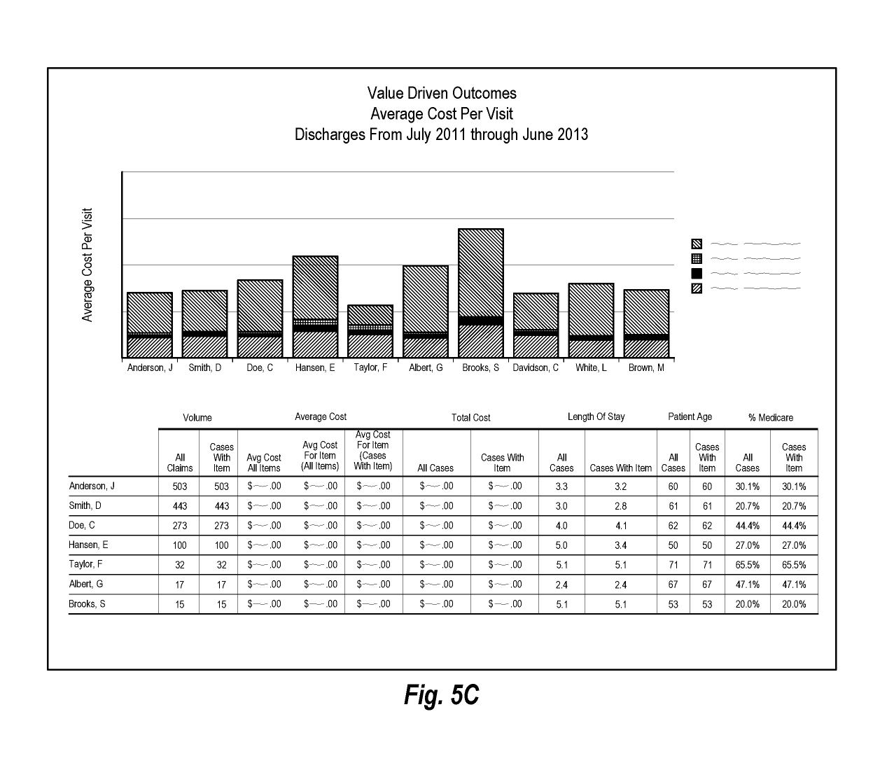

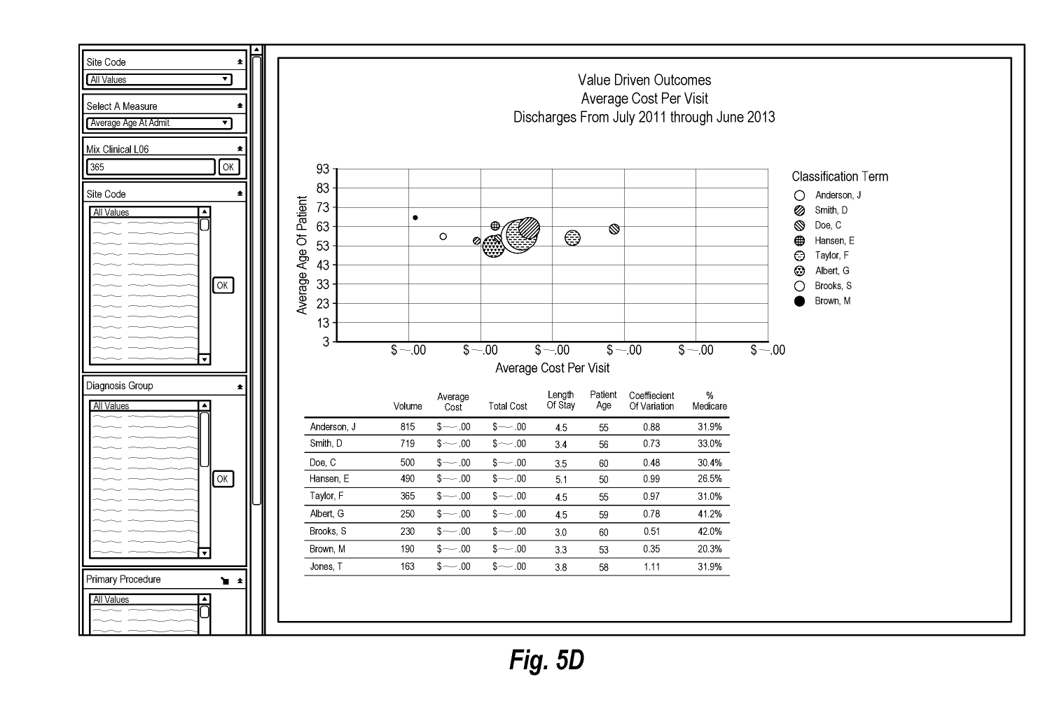

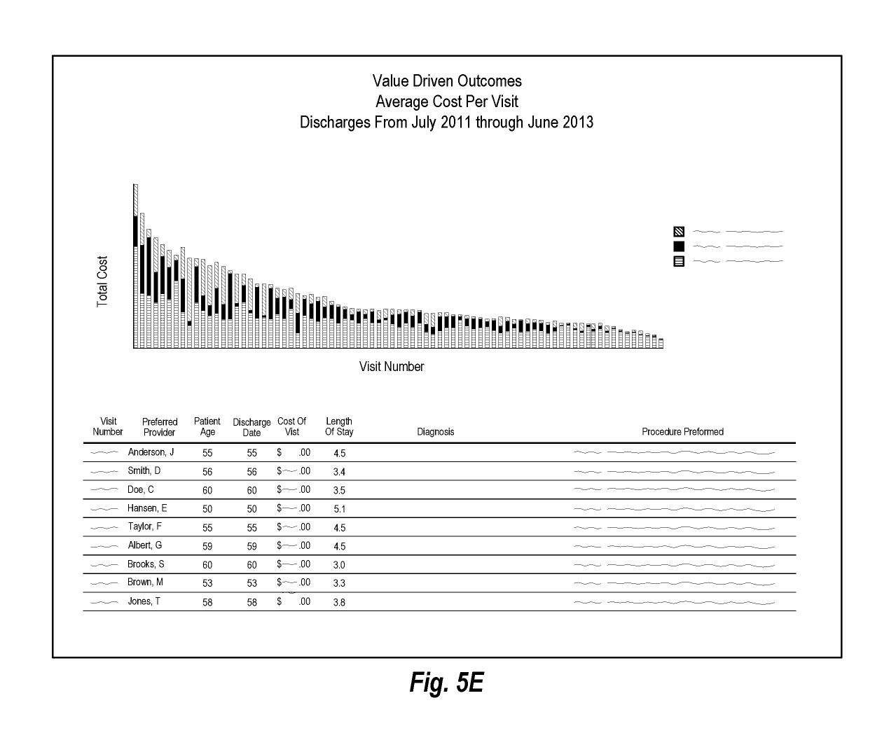

FIGS. 5A-5E illustrate example reports and example dashboards that may be generated by the system 100 of FIG. 1. The reports and/or dashboards of FIGS. 5A-5E may be the reports 108, 158 displayed to the users 104, 154 discussed elsewhere in this disclosure. The reports and/or dashboards may be generated and/or hosted at the agent 200, the multi-tenant cloud 300, or both. The reports and/or dashboards of FIGS. 5A-5E may be customized and/or configured based on a variety of circumstances. For example, the circumstances can include the identity of a user, an employment status of a user, a user's relationship with a health services provider (e.g., patient, physician, medical staff, administrative employee, quality manager, director, accounting employee, IT staff, etc.), a location of the user (e.g., on-premises or off-premises, etc.), a network that a user device is connected to (internal, external, global, firewall secured, etc.), a data requested by a user, and/or other circumstances. The system 100 described with reference to FIG. 1 may generate reports and/or dashboards of one or more of FIGS. 5A-5E. For example, the system 100 may generate reports and/or dashboards based on a request of a user for a report of a certain type or on the circumstances described elsewhere in this disclosure.

In some examples, the types of reports and/or dashboards generated by the system 100 may include an opportunity identification report. FIGS. 5A-5E illustrate an example opportunity identification report. Although the FIGS. 5A-5E illustrate one example configuration of a report, reports may be configured in any suitable manner and may include any suitable information displayed in any suitable manner. In other report configurations, data may be omitted and/or other data may be included and/or displayed in other manners. Each of FIGS. 5A-5E is described in further detail.

FIG. 5A includes outcomes measures and/or composite measures that may be included in the opportunity identification report. Users (e.g., physicians, decision support team members, senior leaders, service line directors, etc.) may select certain outcomes measures to be tracked for each project. For example, certain outcomes measures may be selected if the outcome measures are considered relevant to patients' experiences. In some circumstances, the outcomes measures may be binary (e.g., pass or fail), and the binary outcomes measures may be grouped into a composite measure. If all of the included outcomes measures for a given patient passed, then the composite measure may be determined to be satisfied. If any of the included outcomes measures for a given patient failed, then the composite measure would be determined to be failed. The reports and/or dashboards may display whether the composite measure was passed and/or failed for given circumstances.

The opportunity identification report may include a total direct cost. The total direct cost may be the total facility direct cost, excluding physician fees. The total direct cost may be the sum of the following cost categories: facility utilization costs, supply costs, imaging costs, pharmacy costs, lab costs, and/or other services costs.

The opportunity identification report may also include an opportunity index to identify opportunity. The opportunity index may be defined as procedures where there exist large variations in cost. Generating the opportunity index may include calculating a coefficient of variation, then using a relative rank to rank procedure.

As illustrated in FIG. 5A, the opportunity identification report may include a table displaying the opportunity index versus, for example, a classification term, direct cost, visit count, total cost, performing provider count, coefficient of variation, relative rank and/or other metrics. The table may be sorted in a descending order based on the Relative Rank score. The opportunity identification report may include terms used to classify and/or sort the displayed data. For example, the data may be classified and/or sorted by diagnosis, procedure category, case types, Diagnosis-Related Group (DRG) or International Classification of Diseases, or others. The opportunity identification report may display classification terms: that are the most common, have the highest total costs, and/or have the largest coefficient of variation for costs across attending physicians.

Turning to FIG. 5B, in some configurations the opportunity identification report may include a graphical representation of the data displayed in the table of FIG. 5A. As illustrated, in some configurations, the opportunity identification report may include hover-enabled bubbles representing case types, with bubble sizes reflecting the magnitude of the opportunity.

As illustrated for example in FIG. 5B, the opportunity identification report may include a graphical representation of the coefficient of variation on the y-axis and the average cost per visit on the x-axis. In some configurations, an indicator of the bubbles on the graph may match a corresponding indicator of a procedure, as listed in the legend. The size of the bubble may represent visit count, so a larger bubble may indicate a greater opportunity. As illustrated, in some configurations the opportunity identification report may include a table below the graphical representation indicating, for example, visit level information sorted by provider, volume, average cost per case, total cost, length of stay, average patient age, average coefficient of variation and/or percentage of patients whose primary payor is Medicare. This may permit for identifying by physicians and/or procedures.

As illustrated for example in FIG. 5C, the opportunity identification report may include a graphical representation of average cost per case by each physician who has performed a selected procedure. The graphical representation may indicate cost broken out by the average for each of the cost sub-categories. This graphical representation may be capable of user manipulation. For example, a user may be able to click on a part of the graphical representation or a category in the legend so additional relevant information is displayed. The graphical representation may permit users to see a variation by physician and/or the root of this variation (e.g., in this case, supply cost). As illustrated, in some configurations the opportunity identification report may include a table below the graphical representation indicating, for example, detailed information for costs such as average cost for all cases and/or average cost for cases with a specific item. The specific item may be used for supply analysis, for example, to see the overall average and/or the average for cases with a specific supply item used. This may facilitate identification of supplies that drive sharp increases in cost.

As illustrated for example in FIG. 5D, the opportunity identification report may include a graphical representation (e.g., a scatterplot), where the y-axis is a chosen outcome metric and the x-axis is the average cost per visit with providers indicated with bubbles. The size of the bubble may graphically represent the magnitude of the chosen outcome versus for different providers. This graphical representation may provide context and understanding of patient population, outcomes by provider, and/or chosen outcome metrics.

As illustrated for example in FIG. 5E, the opportunity identification report may include a first graphical representation of the cost of each individual visit, with the visit number on the x-axis and the total cost on the y-axis, sorted by most to least expensive. As illustrated, the opportunity identification report may include a second graphical representation underneath the first graphical representation. The first graphical representation may indicate total direct costs, while the second graphical representation may include underlying cost sub-categories of the total direct cost. In some configurations, users may manipulate the graph to show additional granular levels of data. The tables below each of the first and second graphical representation may indicate the visit number, provider, age, discharge date, cost of the visit, length of stay, diagnosis and/or procedure performed.

In some configurations, reports may be filtered for individual physicians or based on individual outcomes included in the report.

In non-illustrated configurations, reports may be generated for specific procedures, for example, joint replacement surgeries. Such reports may include, for example, average cost per visit, individual outcomes, and/or effects of improved quality versus cost. The outcomes variables for such reports may include, for example: 30 Day Readmit Rate, discharged to OTSS, SCIP Fallout, Early Mobility, HAC/PSI Rate, Return to ED Within 90 Days of Discharge. Such configurations may include a Quality Index of the percentage of visits for that month that satisfied a selected care metric.

The 30 Day Readmit Rate may be the rate of patients who were discharged who were readmitted as inpatients within 30 days of being discharged. The Early Mobility Rate may be the percentage of patients who received a consult from a Physical Therapist on the same day as their joint replacement surgery. The SCIP Fallout Rate may be a measure the health services provider is required to report on. The Discharge to OTSS may be whether or not the patient discharged from the Post Anesthesia Care Unit (PACU) to the Ortho Trauma Surgical Specialties (OTSS). The HAC/PSI rate may be Hospital Acquired Condition (HAC) and Patient Safety Indicators (PSI). Rate of ED Visit Within 90 Days of Discharge may be the rate of patients who visited the Emergency Department within 90 days of being discharged after their surgery.

The reports may include histograms indicating, for example, the distribution of costs based on selected bins. The x-axis may indicate Histogram Bin Number, which corresponds to an actual cost range. The y-axis may indicate the frequency of visits that fall in that bin. The histogram may include a line indicating a running cumulative total.

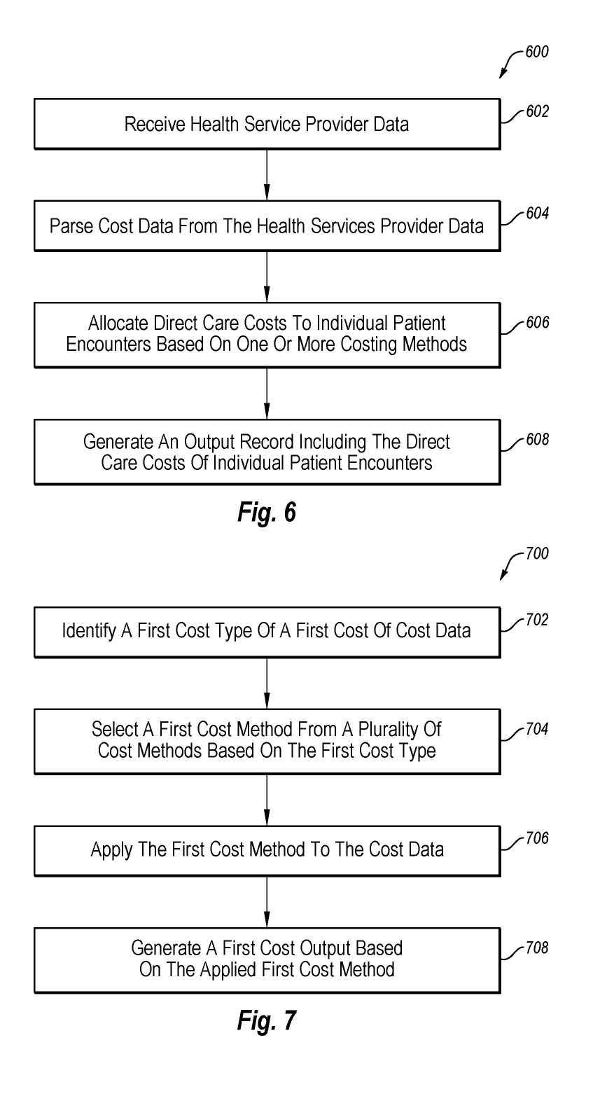

FIG. 6 is a flow chart of an example method 600 of healthcare data management arranged according to at least one embodiment described in this disclosure. The method 600 may be provided at the agent 200 (e.g., at the analysis module 240, etc.) of FIG. 1 and/or at the multi-tenant cloud 300 (e.g., at the shared services 310 and/or at the analysis module 340 of the tenant instance 350, etc.). The method 600 may be stored in memory such as a non-transitory computer readable medium and/or executed by one or more processors such as at the agent 200, the multi-tenant cloud 300, and/or the tenant instance 350. The method 600 may be performed, for example, by the system 100 or a portion of the system 100. Although illustrated as discrete blocks, various blocks may be divided into additional blocks, combined into fewer blocks, or eliminated, depending on the desired implementation.

The method 600 may begin at block 602, in which health service provider data may be received. At block 604, cost data may be parsed from the health service provider data.

At block 606, direct care costs may be allocated to individual patient encounters. For example, in some embodiments, the direct care costs may be allocated based on one or more costing methods. At block 608, an output record may be generated. The output record may include the direct care costs of individual patient encounters.

In some configurations, the generating an output record may include grouping output records and/or cost outputs in groupings. In some configurations, groupings may facilitate displaying and/or reporting grouping output records and/or cost outputs. In some configurations, groupings may include different levels with different details of cost outputs.

One skilled in the art will appreciate that, for this and other procedures and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the disclosed embodiments.

FIG. 7 is a flow chart of an example method 700 of direct care cost allocation arranged in accordance with at least one embodiment discussed in this disclosure. The method 700 may be provided at the agent 200 (e.g., at the analysis module 240, etc.) of FIG. 1 and/or at the multi-tenant cloud 300 (e.g., at the shared services 310 and/or at the analysis module 340 of the tenant instance 350, etc.). The method 700 may be stored in memory such as a non-transitory computer readable medium and/or executed by one or more processors such as at the agent 200, the multi-tenant cloud 300, and/or the tenant instance 350. The method 700 may be performed, for example, by the system 100 or a portion of the system 100. Although illustrated as discrete blocks, various blocks may be divided into additional blocks, combined into fewer blocks, or eliminated, depending on the desired implementation.

The method 700 may begin at block 702 in which a first cost type of a first cost of the cost data may be identified. At block 704, a first cost method may be selected from multiple cost methods. Selection of the first cost method may be based on the first cost type.

At block 706, the first cost method may be applied to the cost data. At block 708, a first cost output may be generated. For example, the first cost output may be based on the applied first cost method. The method 700 may be implemented with other operations or other methods discussed in this disclosure. For instance, in some embodiments, the method 700 may be an example of direct care costs allocation in block 606 of method 600.

In some configurations of the method 700, a cost type may be a grouping of similar expenses into a category that users want to identify at the patient level. Cost types may include fixed direct costs, billable supply costs, depreciation, equipment repair, equipment maintenance, technician labor, staff labor, and/or other cost types. Whether the cost type will affect the first cost output may be determined. A cost flag may be assigned to the first cost output based on whether the first cost type will affect the first cost output.

In some configurations of the method 700, a cost driver for a cost type may be identified. A costing method may be selected from the costing methods based on the cost type and/or the cost driver. The method 700 may include identifying whether a cost is a direct cost or an indirect cost and configuring the costing methods based on whether the cost is a direct or indirect cost.

In some configurations of the method 700, a health services provider unit may be assigned to the first cost of the cost data. Assigning a health services provider unit may include determining a health services provider unit providing a patient service. Additionally, assigning a health services provider unit may include determining the health services provider unit responsible for the first cost. A cost method may be selected and configured based on the assigned health services provider unit. Whether a patient corresponding to a health services provider unit has utilized a service relating to the first cost may be determined and a cost method may be selected and configured based on whether the patient corresponding to the health services provider unit has utilized the service relating to the first cost.

In some configurations of the methods 600, 700, the sources of health service provider data may include accounting data with top-down approach expenses and/or bottom-up approach expenses. The top-down approach expenses may be general operation and/or institutional expenses for the health service provider. The bottom-up approach expenses may be health service provider expenses for which a cost per unit is already known (e.g., purchase price for a lab service, a pharmacy product, a supply resource, etc.). The sources of health service provider data may include costs recorded in the general ledgers of a health service provider and/or associated entities.

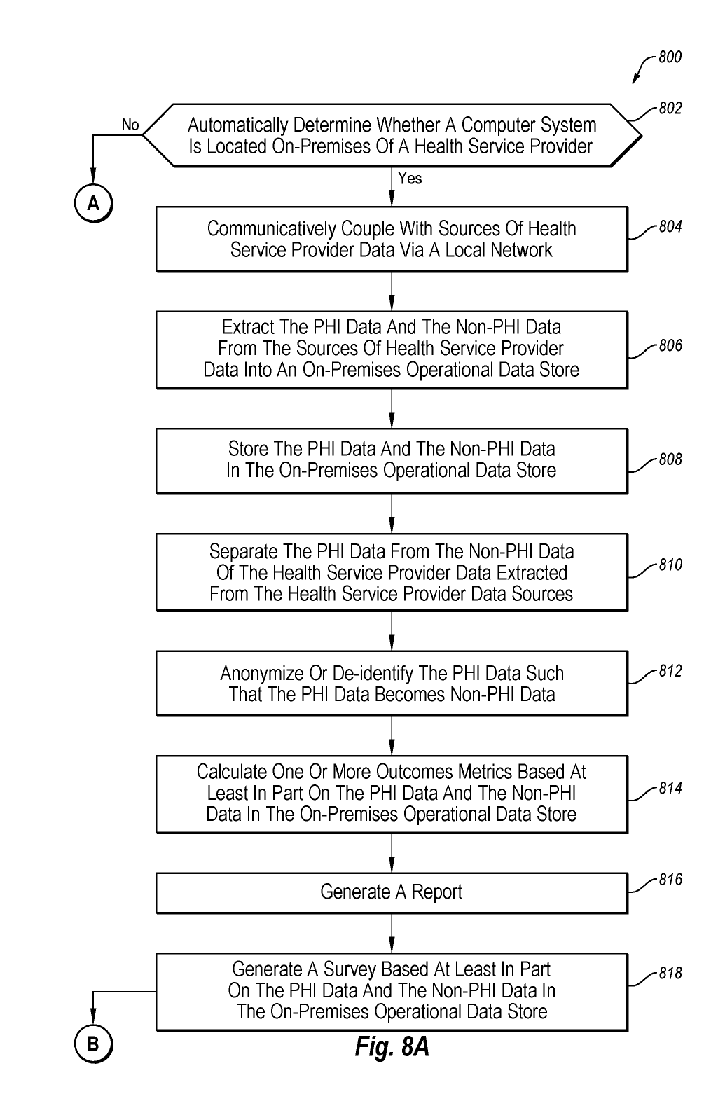

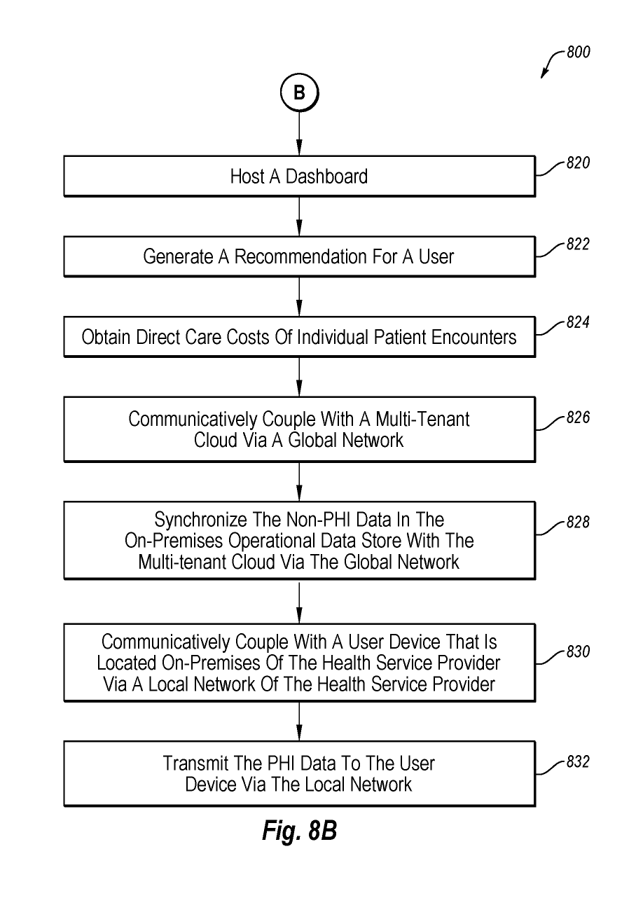

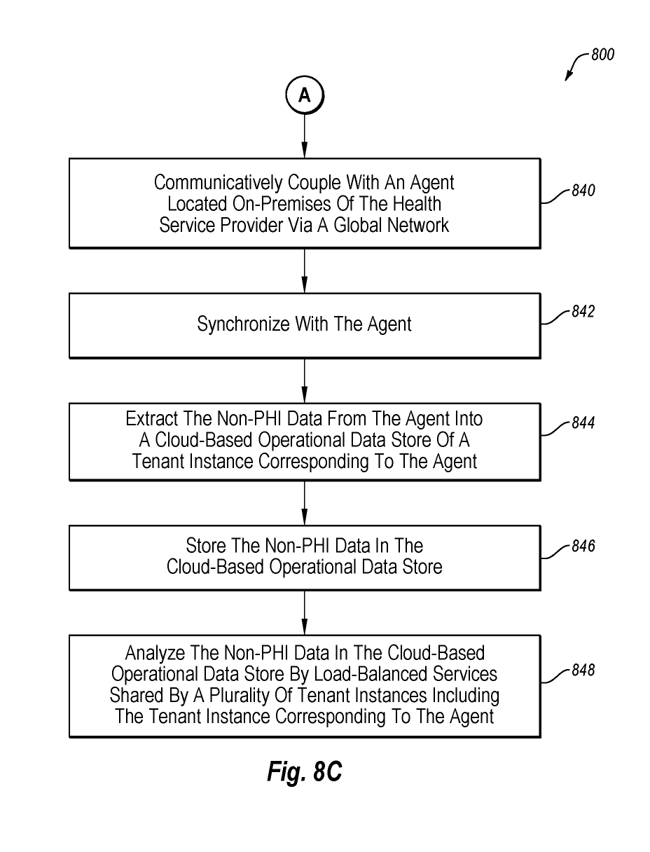

FIGS. 8A-8C are a flow chart of another example method 800 of healthcare data management arranged in accordance with at least one implementation discussed in this disclosure. The method 800 may be provided at the agent 200 (e.g., at the analysis module 240, etc.) of FIG. 1 and/or at the multi-tenant cloud 300 (e.g., at the shared services 310 and/or at the analysis module 340 of the tenant instance 350, etc.). The method 800 may be stored in memory such as a non-transitory computer readable medium and/or executed by one or more processors such as at the agent 200, the multi-tenant cloud 300, and/or the tenant instance 350. The method 800 may be performed, for example, by the system 100 or a portion of the system 100 stored on-premises 102 or off-premises 152. Although illustrated as discrete blocks, various blocks may be divided into additional blocks, combined into fewer blocks, or eliminated, depending on the desired implementation.

With reference to FIG. 8A, the method 800 may begin at block 802. At block 802, it may be determined whether a computer system is located on-premises of a health service provider. Additionally, it may be determined whether the computer system is located in a multi-tenant cloud. In response to a determination that the computer system is located on-premises of a health service provider (“YES” at block 802), the method 800 may proceed to block 804. In response to a determination that the computer system is not located on-premises (“NO” at block 802), the method 800 may proceed to block 840. In some embodiments, the method 800 may proceed to block 840 in response to a determination that the computer system is located in the multi-tenant cloud.

In some embodiments, the determination at block 802 may be effectuated by a computer system carrying out the method 800. In such embodiments, at block 802, the computer system carrying out the method 800 may determine whether it is located on-premises of a health service provider. The computer system may determine that it corresponds to the agent 200 of FIG. 1 in response to a determination that it is located on-premises. The computer system may determine that it corresponds to the multi-tenant cloud 300 of FIG. 1 in response to a determination that it is not located on-premises. In some configurations of the method 800, the determining of the position of the health service provider may be based on one or more of a user input, interfaces coupling the computer system, computer system identifiers, locating identifiers, a network coupled to the computer system, a firewall securing the computer system, or some combination thereof.

At block 804, sources of health service provider data may be communicatively coupled with via a local network. For example, the agent 200 of FIG. 1 may communicatively couple with the health service provider data 110 via the health service provider network 250. At block 806, the health service provider data may be extracted from the health service provider data sources (e.g., the health service provider data 110 of FIG. 2A) into an on-premises operational data store sources (e.g., the operational data store 230 of FIG. 2A). In some embodiments, the health service provider data may include PHI data (e.g., PHI data 112 of FIG. 1) and non-PHI data (e.g., non-PHI data 334 of FIG. 1).

At block 808, the PHI data and the non-PHI data may be stored in an on-premises operational data store. In some embodiments, the on-premises operational data store may be located on-premises of the health service provider.

At block 810, the PHI data may be separated from the non-PHI data of the health service provider data extracted from the health service provider data sources. For example, the separation may be performed at the data intake and mapping module 220 and/or the HIPAA compliant cloud synchronization module 270 of FIG. 2. At block 812, the PHI data may be anonymized or de-identified such that the PHI data becomes non-PHI data. For example, the anonymization or de-identification may be performed at the data intake and mapping module 220 and/or the HIPAA compliant cloud synchronization module 270 of FIG. 2.

At block 814, one or more outcomes metrics may be calculated based at least in part on the PHI data and the non-PHI data in the on-premises operational data store. At block 816, a report may be generated. In some embodiments, the report may be representative of the PHI data, the direct care costs of the individual patient encounters, and the calculated outcomes metrics. At block 818, a survey may be generated based at least in part on the PHI data and the non-PHI data in the on-premises operational data store.

With reference to FIG. 8B, at block 820, a dashboard may be hosted. In some embodiments the dashboard may be configured to display at least some portions of the PHI data and the non-PHI data. Additionally, the dashboard may permit a user to view, evaluate, and interact with the PHI data and the non-PHI data. At block 822, a recommendation for a user may be generated based at least in part on the PHI data and the non-PHI data in the on-premises operational data store.

At block 824, direct care costs of individual patient encounters may be obtained. For example, the direct care costs may be obtained based on the PHI data and the non-PHI data stored in the on-premises operational data store. In some embodiments, the obtaining direct care costs of individual patient encounters may include allocating the direct care costs to the individual patient encounters. In some embodiment, the blocks 814, 816, 818, 820, 822, 824, or some combination thereof may be performed by the analysis module 240 of the agent 200 of FIG. 2A.

At block 826, the method 800 may include communicatively coupling with a multi-tenant cloud via a global network. For example, the agent 200 of FIG. 1 may communicatively couple with the multi-tenant cloud 300 via the network 150. At block 828, the non-PHI data in the on-premises operational data store may be synchronized with the multi-tenant cloud via the global network.

At block 830, the method 800 may include communicatively coupling with a user device that is located on-premises of the health service provider via a local network of the health service provider. For example, the agent 200 of FIG. 2A may be communicatively coupling with the user device 106a via the health service provider network 250. At block 832, the PHI data may be transmitted to the user device via the local network. For example, the PHI data 112 of FIG. 2A may be transmitted to the user device 106a via the health service provider network 250.

With reference to FIG. 8C, at block 840, the method 800 may include communicatively coupling with an agent located on-premises of the health service provider. The coupling may be via a global network. At block 842, synchronization with the agent may be performed. The synchronization may include receiving the non-PHI data absent the PHI data from the agent via the global network. At block 844, the non-PHI data may be extracted from the agent into a cloud-based operational data store of a tenant instance. The tenant instance may correspond to the agent. At block 846, the non-PHI data may be stored in the cloud-based operational data store. At block 848, the non-PHI data may be analyzed in the cloud-based operational data store. The analysis may be performed by load-balanced services shared by two or more tenant instances. The two or more tenant instances may include the tenant instance corresponding to the agent.

FIG. 9 is a flow chart of an example method 900 in accordance with at least one implementation discussed in this disclosure. The method 900 may be provided at the agent 200 (e.g., at the analysis module 240, etc.) of FIG. 1 and/or at the multi-tenant cloud 300 (e.g., at the shared services 310 and/or at the analysis module 340 of the tenant instance 350, etc.). The method 900 may be stored in memory such as a non-transitory computer readable medium and/or executed by one or more processors such as at the agent 200, the multi-tenant cloud 300, and/or the tenant instance 350. The method 900 may be performed, for example, by the system 100 or a portion of the system 100 stored on-premises 102 or off-premises 152. Although illustrated as discrete blocks, various blocks may be divided into additional blocks, combined into fewer blocks, or eliminated, depending on the desired implementation.

With reference to FIG. 9, the method 900 may begin at block 902. At block 902, health service provider data sources may be coupled with. For example, the agent 200 of FIG. 1 may be coupled with the health service provider data sources. In some embodiments, the health service provider data may include accounting data, clinical data, and electronic health records. At block 904, health service provider data may be extracted from the health service provider data sources. The health service provider data may include PHI data and non-PHI data. At block 906, the PHI data and the non-PHI data may be stored in an on-premises operational data store.

At block 908, direct care costs of individual patient encounters may be obtained. In some implementations, the obtaining may be performed based on the PHI data and the non-PHI data in the on-premises operational data store. The obtaining the direct care costs may include parsing cost data from the PHI data and the non-PHI data in the on-premises operational data store. Additionally or alternatively, the obtaining the direct care costs may include allocating direct care costs to individual patient encounters based on one or more costing methods. Additionally or alternatively, the obtaining the direct care costs may include generating an output record that includes the direct care costs of individual patient encounters. For example, with reference to FIGS. 6 and 7, the obtaining the direct care costs may include one or more of steps 602, 604, 606, 608, 702, 704, 706, 708, or some combination thereof.

At block 910, the method 900 may include communicatively coupling with a health service provider tenant instance of a multi-tenant cloud via a global network. At block 912, the non-PHI data in the on-premises operational data store may be synchronized with the multi-tenant cloud via the global network. At block 914, a user device located on-premises of the health service provider may be communicatively coupled with via a local network of the health service provider.

At block 916, it may be determined whether a first user on-premises of the health service provider is authorized to view PHI data. In response to a determination that the first user is authorized to view PHI data (“YES” at block 916), the method 900 may proceed to block 918. In response to a determination that the first user is not authorized to view PHI data (“NO” at block 916), the method 900 may proceed to block 920. At block 918, the PHI data may be transmitted to the user device via the local network. At block 920, non-PHI data absent of the PHI data may be transmitted to the user device via the local network.

In some configurations, the method 900 may include determining whether extracted health service provider data includes the PHI data or the non-PHI data. The method may further include separating PHI data from non-PHI data and/or rendering the PHI data as non-PHI data by anonymizing or de-identifying the PHI data in response to a determination that the data extracted from the sources of the health service provider data is PHI data.

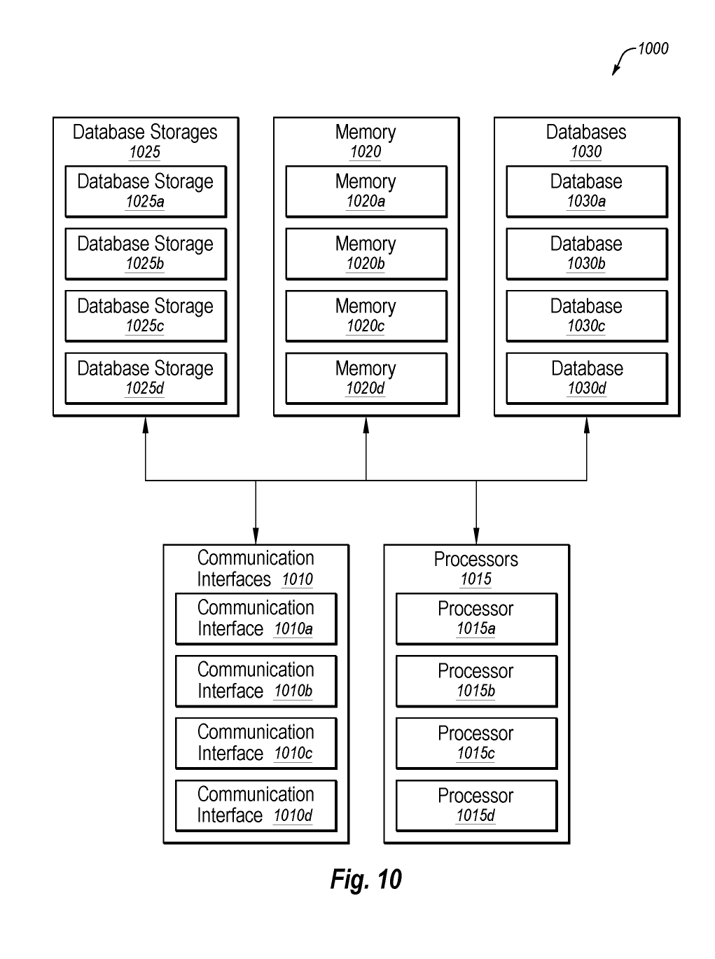

FIG. 10 illustrates a representation of an example computing system 1000 that may be implemented for value driven outcomes. For example, the agent 200 of FIG. 2A and/or the multi-tenant cloud 300 of FIGS. 3A and 3B may include one or more computing systems such as the computing system 1000.

The computing system 1000 may include one or more communication interfaces 1010, one or more processors 1015, one or more memory 1020, one or more data storages 1025, one or more databases 1030, or some combination thereof. The communication interfaces 1010, processors 1015, memory 1020, data storages 1025, and databases 1030 may be communicatively coupled to one another in any suitable configuration.

The communication interfaces 1010 may permit the synchronization module 270 of FIG. 2A to communicate via the network 150 and/or the user interface module 260 of FIG. 2A to communicate via the health service provider network 250, for example. The communication interfaces 1010 may permit communication using any communication protocol, interface, standard, etc. In some embodiments, the communication interfaces 1010 may permit communication using a wired or a wireless connection.

The processors 1015 may interpret and/or execute program instructions and/or process data stored in the memory 1020, the data storages 1025, the databases 1030, or some combination thereof. In some embodiments, the processors 1015 may fetch program instructions from the data storages 1025 and/or the databases 1030 and load the program instructions in the memory 1020. After the program instructions are loaded into the memory 1020, the processors 1015 may execute the program instructions.

For example, program instructions may include operations included in of one or more of the methods 600, 700, 800, and 900. The processors 1015 may access the program instructions and perform or control performance of one or more of the methods 600, 700, and 800.

The processors 1015 may include any suitable special-purpose or general-purpose computer, computing entity, or processing device including various computer hardware or software modules and may be configured to execute instructions stored on any applicable computer-readable storage media. For example, a processor may be a microprocessor, a microcontroller, a digital signal processor (DSP), an application-specific integrated circuit (ASIC), a field-programmable gate array (FPGA), or any other digital or analog circuitry configured to interpret and/or to execute program instructions and/or to process data. The processor may interpret and/or execute program instructions and/or process data stored in memory. The processor may fetch program instructions from and load the program instructions in memory. After the program instructions are loaded into memory, the processor may execute the program instructions.

The memory 1020 and the data storages 1025 may include computer-readable storage media for carrying or having computer-executable instructions or data structures stored thereon. The computer-readable storage media may be any available media that may be accessed by a computing system and/or a processor (e.g., 1015). The computer-readable storage media may include tangible and/or non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium that may be used to carry or store desired program code in the form of computer-executable instructions or data structures and that may be accessed by a general-purpose or special-purpose computer. Any combinations of the above may also be included within the scope of computer-readable storage media.

The databases 1030 may also include multiple modules, that when executed by the processors 1015, may cause the processors 1015 to perform operations, such as described elsewhere in this disclosure. In some embodiments, the databases 1030 may include computer-readable storage media for carrying or having computer-executable instructions or data structures stored thereon. Computer-executable instructions may include, for example, instructions and data configured to cause the processors 1015 to perform a certain operation or group of operations.

The computing system 1000 may be a load balanced computing system. The communication interfaces 1010, the processors 1015, the memory 1020, the data storages 1025, and the databases 1030 may be load balanced shared resources (collectively referred to as “shared resources”). In some configurations, the shared resources may be used to generate virtual machines, virtual databases, virtual private servers, system virtual machines, process virtual machines, and the like. Such configurations may facilitate persistent access to the shared resources. The computing system 1000 may include software and/or algorithms stored in memory to be executed by a processor to load balance the shared resources based on the demand for the shared resources. Load balancing may include distributing computing workloads and/or data storage needs across the shared resources. Load balancing may optimize and/or prioritize resource use, maximize throughput, minimize response time, and/or avoid overload of any single resource. The computing system 1000 may be configured to load balance with multiple resources to increase reliability through redundancy. The computing system 1000 may include a virtual database manager (“VDB”) including software stored in memory, that when executed, for example, by a processor, may represent non-relational data in virtual data warehouses.

With combined reference to FIGS. 3A, 4A and 10, in configurations in which the computing system 1000 is implemented in the multi-tenant cloud 300, the shared resources may be used by the multi-tenant cloud 300 to provide the shared services 310. The shared resources may be used to generate virtual machines corresponding to the tenant instances 350. The tenant management module 304 may be configured to load balance the shared resources of the computing system 1000 for the multi-tenant cloud 300. The tenant management module 304 may be configured to distribute the shared resources of the computing system 1000 across the tenant instances 350. Additionally, the databases 1030 may be used to generate virtual databases corresponding to the ODS 330 of the tenant instances 350.

The embodiments described in this disclosure may include the use of a special purpose or general-purpose computer including various computer hardware or software modules, as discussed in greater detail below.

Embodiments within the scope of this disclosure also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used in this disclosure, the term “module” or “component” may refer to software objects or routines that execute on the computing system. The different components, modules, engines, and services described herein may be implemented as objects or processes that execute on the computing system (e.g., as separate threads). While the system and methods described herein may be implemented in software, implementations in hardware or a combination of software and hardware are also possible and contemplated. In this description, a “computer” may be any computing system as previously defined herein, or any module or combination of modulates running on a computing system.

As used in this disclosure, the term “automatically” may refer to: one or more tasks accomplished without interaction by a user; one or more tasks accomplished without user interaction after activation by a user; and/or one or more tasks accomplished by a computer system with little or no direct human control.

For the processes and/or methods disclosed, the functions performed in the processes and methods may be implemented in differing order, as may be indicated by context. Furthermore, the outlined steps and operations are only provided as examples, and some of the steps and operations may be optional, combined into fewer steps and operations, or expanded into additional steps and operations.

This disclosure may sometimes illustrate different components contained within, or connected with, different other components. Such depicted architectures are merely exemplary, and many other architectures can be implemented which achieve the same or similar functionality.

The terms used in this disclosure, and in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.). In addition, if a specific number of elements is introduced, this may be interpreted to mean at least the recited number, as may be indicated by context (e.g., the bare recitation of “two recitations,” without other modifiers, means at least two recitations, or two or more recitations). As used in this disclosure, any disjunctive word and/or phrase presenting two or more alternative terms should be understood to contemplate the possibilities of including one of the terms, either of the terms, or both terms. For example, the phrase “A or B” will be understood to include the possibilities of “A” or “B” or “A and B.”

The terms and words used are not limited to the bibliographical meanings, but, are merely used to enable a clear and consistent understanding of the disclosure. It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to those skilled in the art, may occur in amounts that do not preclude the effect the characteristic was intended to provide.

Aspects of the present disclosure may be embodied in other forms without departing from its spirit or essential characteristics. The described aspects are to be considered in all respects illustrative and not restrictive. The claimed subject matter is indicated by the appended claims rather than by the foregoing description. All changes which come within the meaning and range of equivalency of the claims are to be embraced within their scope.

What is claimed is:

1. A method for providing either enhanced data access functionality or reduced data access functionality to a secured network entity based on a determined current geographic location of a computer system implementing the method and whether the computer system's current geographic location is within a specified area, the method comprising:
automatically determining whether the computer system is located on-premises of a health service provider or on a multi-tenant cloud, the determination including identifying a status of the computer system as being on-premises or off-premises based on the computer system's current geographic location as measured using one or more hardware-based network interfaces configured to pinpoint the computer system's current geographic location;
based the computer system's status of being either on-premises or off-premises as determined by the one or more hardware-based network interfaces, activating or deactivating access to an agent interface that facilitates transmission of health service provider data to the computer system, wherein access to the agent interface is activated when the computer system's status is indicated as being on-premises and is deactivated when the computer system's status is indicated as being off-premises such that different levels of access to the health service provider data are provided to the computer system depending on where the computer system is physically located;
in response to a determination that the computer system's status is indicated as being on-premises, activate access to the agent interface and perform at least the following:
communicatively coupling with one or more health service provider data sources via a local network;
instantiating one or more machine learning algorithms to identify one or more types of interfaces used by the provider data sources and/or one or more data storage formats used by the provider data sources;
extracting health service provider data from the health service provider data sources using the interfaces and/or data storage formats identified using the machine learning algorithms, wherein the health service provider data includes data categorized as protected health information (PHI) data and data categorized as non-PHI data;
storing the data categorized as PHI data and the data categorized as non-PHI data in an on-premises operational data store that is located on-premises of the health service provider;
obtaining data analytics based on the data categorized as PHI data and the data categorized as non-PHI data stored in the on-premises operational data store;
communicatively coupling with a multi-tenant cloud via a global network; and
synchronizing only the data categorized as non-PHI data in the on-premises operational data store with the multi-tenant cloud via the global network, such that the multi-tenant cloud includes only the data categorized as non-PHI data and other data categorized as non-PHI data from one or more other health service providers, wherein the synchronization is performed in response to the computer system identifying itself as being located on-premises of the health service provider, and wherein the synchronization includes encrypting the data categorized as non-PHI data prior to transfer to the multi-tenant cloud.

2. The method of claim 1, further comprising separating the data categorized as PHI data from the data categorized as non-PHI data of the health service provider data extracted from the health service provider data sources.

3. The method of claim 1, further comprising anonymizing or de-identifying the data categorized as PHI data such that the data categorized as PHI data becomes non-PHI data.

4. The method of claim 1, further comprising calculating one or more data analytics results based at least in part on the data categorized as PHI data and the data categorized as non-PHI data in the on-premises operational data store.

5. The method of claim 4, further comprising one or more or a combination of:
generating a report representative of the data categorized as PHI data and the data analytics results;
generating a survey based at least in part on the data categorized as PHI data and the data categorized as non-PHI data in the on-premises operational data store;
hosting a dashboard that displays at least some portions of the data categorized as PHI data and the data categorized as non-PHI data and to permit a user to view, evaluate, and interact with the data categorized as PHI data and the data categorized as non-PHI data; and
generating a recommendation for a user based at least in part on the data categorized as PHI data and the data categorized as non-PHI data in the on-premises operational data store.

6. The method of claim 1, further comprising:
communicatively coupling with a user device that is located on-premises of the health service provider via a local network of the health service provider; and
transmitting the data categorized as PHI data to the user device via the local network.

7. The method of claim 1, wherein the automatically determining is based on one or more or a combination of: a user input, an interface coupling the computer system, a computer system identifier, a locating identifier, a network coupled to the computer system, and a firewall securing the computer system.

8. The method of claim 1, further comprising in response to a determination that the computer system is located on the multi-tenant cloud:
communicatively coupling with an agent located on-premises of the health service provider via a global network;
wherein the synchronizing includes synchronizing with the agent and receiving non-PHI data absent PHI data from the agent via the global network;
extracting the non-PHI data received from the agent into a cloud-based operational data store of a tenant instance that corresponds to the agent;
storing the non-PHI data in the cloud-based operational data store; and
analyzing the non-PHI data in the cloud-based operational data store by load-balanced services shared by a plurality of tenant instances including the tenant instance that corresponds to the agent.

9. A non-transitory computer readable medium having computer-executable instructions which, when executed by one or more processors, cause the one or more processors to perform or control performance of the method of claim 1.

10. A computer system comprising:
one or more processors; and
one or more computer-readable hardware storage devices having stored thereon computer-executable instructions that are executable by the one or more processors to cause the computer system to provide either enhanced data access functionality or reduced data access functionality to a secured network entity based on a determined current geographic location of the computer system and whether the computer system's current geographic location is within a specified area by causing the computer system to:
automatically determine whether the computer system is located on-premises of a health service provider or on a multi-tenant cloud, the determination including identifying a status of the computer system as being on-premises or off-premises based on the computer system's current geographic location as measured using one or more hardware-based network interfaces configured to pinpoint the computer system's current geographic location;
based the computer system's status of being either on-premises or off-premises as determined by the one or more hardware-based network interfaces, activate or deactivate access to an agent interface that facilitates transmission of health service provider data to the computer system, wherein access to the agent interface is activated when the computer system's status is indicated as being on-premises and is deactivated when the computer system's status is indicated as being off-premises such that different levels of access to the health service provider data are provided to the computer system depending on where the computer system is physically located;
in response to a determination that the computer system's status is indicated as being on-premises, activate access to the agent interface and perform at least the following:
communicatively coupling with one or more health service provider data sources via a local network;
instantiating one or more machine learning algorithms to identify one or more types of interfaces used by the provider data sources and/or one or more data storage formats used by the provider data sources;
extracting health service provider data from the health service provider data sources using the interfaces and/or data storage formats identified using the machine learning algorithms, wherein the health service provider data includes data categorized as protected health information (PHI) data and data categorized as non-PHI data;
storing the data categorized as PHI data and the data categorized as non-PHI data in an on-premises operational data store that is located on-premises of the health service provider;
obtaining data analytics based on the data categorized as PHI data and the data categorized as non-PHI data stored in the on-premises operational data store;
communicatively coupling with a multi-tenant cloud via a global network; and
synchronizing only the data categorized as non-PHI data in the on-premises operational data store with the multi-tenant cloud via the global network, such that the multi-tenant cloud includes only the data categorized as non-PHI data and other data categorized as non-PHI data from one or more other health service providers, wherein the synchronization is performed in response to the computer system identifying itself as being located on-premises of the health service provider, and wherein the synchronization includes encrypting the data categorized as non-PHI data prior to transfer to the multi-tenant cloud.

11. The computer system of claim 10, wherein execution of the computer-executable instructions further causes the computer system to:
separate the data categorized as PHI data from the data categorized as non-PHI data of the health service provider data extracted from the health service provider data sources.

12. The computer system of claim 10, wherein execution of the computer-executable instructions further causes the computer system to:
anonymize or de-identify the data categorized as PHI data such that the data categorized as PHI data becomes non-PHI data.

13. The computer system of claim 10, wherein execution of the computer-executable instructions further causes the computer system to:
calculate one or more data analytics results based at least in part on the data categorized as PHI data and the data categorized as non-PHI data in the on-premises operational data store.

14. The computer system of claim 13, wherein execution of the computer-executable instructions further causes the computer system to perform any one or more of:
generate a report representative of the data categorized as PHI data and the data analytics results;
generate a survey based at least in part on the data categorized as PHI data and the data categorized as non-PHI data in the on-premises operational data store;
host a dashboard that displays at least some portions of the data categorized as PHI data and the data categorized as non-PHI data and to permit a user to view, evaluate, and interact with the data categorized as PHI data and the data categorized as non-PHI data; and
generate a recommendation for a user based at least in part on the data categorized as PHI data and the data categorized as non-PHI data in the on-premises operational data store.

15. The computer system of claim 10, wherein execution of the computer-executable instructions further causes the computer system to:
communicatively couple with a user device that is located on-premises of the health service provider via a local network of the health service provider; and
transmit the data categorized as PHI data to the user device via the local network.

16. The computer system of claim 10, wherein the automatically determining is based on one or more or a combination of: a user input, an interface coupling the computer system, a computer system identifier, a locating identifier, a network coupled to the computer system, and a firewall securing the computer system.

17. The computer system of claim 10, wherein execution of the computer-executable instruction further causes the computer system to, in response to a determination that the computer system is located on the multi-tenant cloud:
communicatively couple with an agent located on-premises of the health service provider via a global network, wherein the synchronizing includes synchronizing with the agent and receiving non-PHI data absent PHI data from the agent via the global network;
extract the non-PHI data received from the agent into a cloud-based operational data store of a tenant instance that corresponds to the agent;
store the non-PHI data in the cloud-based operational data store; and
analyze the non-PHI data in the cloud-based operational data store by load-balanced services shared by a plurality of tenant instances including the tenant instance that corresponds to the agent.

18. One or more hardware storage devices having stored thereon computer-executable instructions that are executable by one or more processors of a computer system to cause the computer system to provide either enhanced data access functionality or reduced data access functionality to a secured network entity based on a determined current geographic location of the computer system and whether the computer system's current geographic location is within a specified area by causing the computer system to:
automatically determine whether the computer system is located on-premises of a health service provider or on a multi-tenant cloud, the determination including identifying a status of the computer system as being on-premises or off-premises based on the computer system's current geographic location as measured using one or more hardware-based network interfaces configured to pinpoint the computer system's current geographic location;
based the computer system's status of being either on-premises or off-premises as determined by the one or more hardware-based network interfaces, activate or deactivate access to an agent interface that facilitates transmission of health service provider data to the computer system, wherein access to the agent interface is activated when the computer system's status is indicated as being on-premises and is deactivated when the computer system's status is indicated as being off-premises such that different levels of access to the health service provider data are provided to the computer system depending on where the computer system is physically located;
in response to a determination that the computer system's status is indicated as being on-premises, activate access to the agent interface and perform at least the following:
communicatively coupling with one or more health service provider data sources via a local network;
instantiating one or more machine learning algorithms to identify one or more types of interfaces used by the provider data sources and/or one or more data storage formats used by the provider data sources;
extracting health service provider data from the health service provider data sources using the interfaces and/or data storage formats identified using the machine learning algorithms, wherein the health service provider data includes data categorized as protected health information (PHI) data and data categorized as non-PHI data;
storing the data categorized as PHI data and the data categorized as non-PHI data in an on-premises operational data store that is located on-premises of the health service provider;
obtaining data analytics based on the data categorized as PHI data and the data categorized as non-PHI data stored in the on-premises operational data store;
communicatively coupling with a multi-tenant cloud via a global network; and
synchronizing only the data categorized as non-PHI data in the on-premises operational data store with the multi-tenant cloud via the global network, such that the multi-tenant cloud includes only the data categorized as non-PHI data and other data categorized as non-PHI data from one or more other health service providers, wherein the synchronization is performed in response to the computer system identifying itself as being located on-premises of the health service provider, and wherein the synchronization includes encrypting the data categorized as non-PHI data prior to transfer to the multi-tenant cloud.

19. The one or more hardware storage devise of claim 18, wherein execution of the computer-executable instructions further causes the computer system to:
separate the data categorized as PHI data from the data categorized as non-PHI data of the health service provider data extracted from the health service provider data sources.

20. The one or more hardware storage devise of claim 18, wherein execution of the computer-executable instructions further causes the computer system to:
anonymize or de-identify the data categorized as PHI data such that the data categorized as PHI data becomes non-PHI data.
